(19) United States
(12) Patent Application Publication
(10) Pub. No.: US 2024/0180931 A1
(43) Pub. Date: Jun. 6, 2024

(54) CANNABINOIDS AND USES THEREOF IN TREATMENT OF A DISEASE

(71) Applicant: TECHNION RESEARCH & DEVELOPMENT FOUNDATION LIMITED, Haifa (IL)

(72) Inventors: Dedi MEIRI, Haifa (IL); Yaron FUCHS, Haifa (IL); Elle KOREN, Haifa (IL); Egor SEDOV, Haifa (IL)

(73) Assignee: TECHNION RESEARCH & DEVELOPMENT FOUNDATION LIMITED, Haifa (IL)

(21) Appl. No.: 18/432,341

(22) Filed: Feb. 5, 2024

Related U.S. Application Data
(63) Continuation of application No. PCT/IL2022/050850, filed on Aug. 4, 2022.
(60) Provisional application No. 63/229,780, filed on Aug. 5, 2021.
(60) Provisional application No. 63/229,787, filed on Aug. 5, 2021.
(60) Provisional application No. 63/229,820, filed on Aug. 5, 2021.

(51) Int. Cl. A61K 31/00; A61K 36/185; A61P 17/02; A61P 17/06; A61P 17/14

(52) U.S. Cl. CPC A61K 31/658; A61K 36/185; A61P 17/02; A61P 17/06; A61P 17/14

(57) ABSTRACT

The present invention provides a pharmaceutical composition including a combination of one or more cannabinoids, and a methods of using same, such as for treating any one of: a wound, regenerating skin, alopecia, an inflammatory skin disease, a sebaceous gland proliferative disease, and any disorder, condition, or symptom associated therewith.

### CROSS-REFERENCE TO RELATED APPLICATIONS

This application is a ByPass Continuation of PCT Patent Application No. PCT/IL2022/050850 having International filing date of Aug. 4, 2022, which claims the benefit of priority of U.S. Provisional Patent Application No. 63/229,780, titled “CANNABINOIDS AND USES IN THEREOF THE TREATMENT OF INFLAMMATORY SKIN DISEASE”, filed on 5 Aug. 2021, No. 63/229,787, titled “CANNABINOIDS AND USES THEREOF FOR TREATMENT OF SEBACEOUS GLAND PROLIFERATIVE DISEASE”, filed on 5 Aug. 2021, and No. 63/229,820, titled “CANNABINOIDS AND USES THEREOF FOR SKIN REGENERATION”, filed on 5 Aug. 2021. The contents of all of these applications are hereby incorporated by reference in their entirety.

### FIELD OF INVENTION

The present disclosure relates to pharmaceutical compositions comprising at least one cannabinoid, methods of use and methods of treatment thereof.

### BACKGROUND

Manipulating the activity of the endocannabinoid system (ECS) offers therapeutic potential for a multitude of diseases and pathological conditions affecting humans. ECS receptors such as cannabinoid receptor 1 (CB1) and cannabinoid receptor 2 (CB2) have been found to be expressed in the skin. Thus, various strains of Cannabis sativa and its derivatives, the cannabinoids, have gained substantial interest as a potential therapeutic avenue for treating inflammatory disease, such as skin inflammatory diseases.

Therefore, there is still a great need for pharmaceutical compositions suitable for treating a subject afflicted with a disease.

### SUMMARY

According to one aspect, there is provided a method for treating any one of: a wound, regenerating skin, alopecia, and any disorder, condition, or symptom associated therewith, in a subject in need thereof, the method comprising administering to the subject a therapeutically effective amount of a pharmaceutical composition comprising one to five cannabinoids, wherein the one to five cannabinoids comprises CBG, thereby treating a wound, regenerating skin, or both, in the subject.

According to another aspect, there is provided a method for treating a subject afflicted with an inflammatory skin disease, the method comprising administering to the subject a therapeutically effective amount of a pharmaceutical composition comprising one to five cannabinoids, wherein the one to five cannabinoids comprises: CBN, thereby treating the subject afflicted with an inflammatory skin disease.

According to another aspect, there is provided a method for treating a subject afflicted with a sebaceous gland proliferative disease, comprising administering to the subject a therapeutically effective amount of a pharmaceutical composition comprising one to five cannabinoids, wherein the one to five cannabinoids comprises: CBN, CBG, CBC, THC, or any combination thereof, thereby treating the subject afflicted with a sebaceous gland proliferative disease.

According to another aspect, there is provided a pharmaceutical composition comprising one to five cannabinoids, wherein the one to five cannabinoids comprises CBG, for use in the treatment of any one of: a wound, skin regeneration, alopecia, any condition, or disorder associated therewith, and any combination thereof.

According to another aspect, there is provided a pharmaceutical composition comprising one to five cannabinoids, wherein the one to five cannabinoids comprises CBN, for use in the treatment of a subject afflicted with an inflammatory skin disease.

According to another aspect, there is provided a pharmaceutical composition comprising one to five cannabinoids, wherein the one to five cannabinoids comprises: CBN, CBG, CBC, THC, or any combination thereof, for use in the treatment of a sebaceous gland proliferative disease.

According to another aspect, there is provided a pharmaceutical composition comprising one to five cannabinoids, wherein the one to five cannabinoids comprises CBG, for use in the treatment of any one of: a wound, skin regeneration, alopecia, any condition, or disorder associated therewith, and any combination thereof.

In some embodiments, the one to five cannabinoids is CBG.

In some embodiments, the pharmaceutical composition further comprises tetrahydrocannabinol (THC).

In some embodiments, the THC is (−)-Δ9-trans-tetrahydrocannabinol (Δ9-THC).

In some embodiments, the CBG and THC are present in the composition in a weight per weight ratio of about 1:10 (w/w) to 1:35 (w/w).

In some embodiments, the pharmaceutical composition is devoid of CBN and CBC.

In some embodiments, one or more of the cannabinoids is present as a highly purified extract of Cannabis.

In some embodiments, one or more of the cannabinoids is a synthetically produced cannabinoid.

In some embodiments, the treating comprises reducing: the area size of the wound, increasing the number of skin invaginations, increasing the number of skin proliferating cells, increasing the proliferation rate of skin cells, inducing hair follicle growth or regeneration, inhibiting, or reducing scarring, or any combination thereof, in the subject.

In some embodiments, the one to five cannabinoids is CBN.

In some embodiments, the one to five cannabinoids comprises THC.

In some embodiments, the pharmaceutical composition comprises CBN and THC in a weight per weight ratio (w/w) ranging from 1:10 to 1:40.

In some embodiments, the pharmaceutical composition is devoid of any one of: CBG, CBC, and both.

In some embodiments, the treating comprises inhibiting or reducing: NF-κB migration from the cytosol to the nucleus, NF-κB signaling, % of nuclear NF-κB, or any combination thereof, in keratinocytes of the subject.

In some embodiments, the inflammatory skin disease comprises a keratinocytes-related inflammatory disease.

In some embodiments, the inflammatory skin disease is selected from the group consisting of: psoriasis, allergic contact dermatitis, and atopic dermatitis.

In some embodiments, the pharmaceutical composition comprises CBN, CBG, CBC, and THC.

In some embodiments, the pharmaceutical composition comprises any one of: (a) CBN and CBG in a weight per weight ratio (w/w) ranging from 2:1 to 1:2; (b) CBN and CBC in a w/w ranging from 5:1 to 1:1; (c) CBG and CBC in a w/w ranging from 4:1 to 2:1; (d) CBN and THC in a w/w ranging from 1:5 to 1:35; or (e) any combination of (a) to (d).

In some embodiments, the treating comprises reducing: the number, rate, or both, of proliferating sebaceous cells, the size, growth rate, or both of sebaceous glands, sebum amount, production rate, or both, or any combination thereof, in the subject.

In some embodiments, the sebaceous gland proliferative disease is a hyperproliferative disease.

In some embodiments, the sebaceous gland proliferative disease is selected from the group consisting of: acne, sebaceous carcinoma, sebaceous hyperplasia, seborrhoea, seborrhoeic dermatitis, sebopsoriasis, sebaceous adenoma, nevus sebaceous, or phymatous rosacea.

In some embodiments, the pharmaceutical composition is a tablet, a capsule, a gel, a semi-liquid, a liquid, a solution, an emulsion, a spray, a chewable food product, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D include a schematic illustration, a table, fluorescent micrographs, and a vertical bar graph, showing that the cannabis strain Infinity (INF) prevents psoriasis development in a murine skin. (1A) A schematic non-limiting study design showing acute 5%-Imiquimod (IMQ)-mediated psoriasis and treatments regimen in vivo. (1B) Table outlining cannabis strains. (1C) Immunostaining of treated psoriatic skins (day 8) for epidermal marker Krt5. While dotted lines demarcate uppermost layer of the epidermis. (1D) Quantification of epidermal width after 8 days. ***p<0.001 compared to the control as determined by two-tailed Student's T test. Error bars show S.E.M. n<3 in all experiments.

FIGS. 2A-2D include fluorescent micrographs and a vertical bar graph showing that INF blocks symptoms of psoriasis including excessive proliferation, differentiation defects and inflammation. (2A) Immunostaining of treated psoriatic skins (day 8) for proliferation marker Mcm2. (2B) Quantification of proliferating epidermal cells after treatment. ns=no significance as determined by two-tailed Student's T test. Error bars show S.E.M. (2C-2D) Psoriatic skins immunostained against (2C) basal cell marker Krt15 and (2D) inflammation marker tumor necrosis factor alpha (TNFα). All white dotted lines demarcate epidermis/dermis boundary. n<3 in all experiments.

FIGS. 3A-3D include a table, fluorescent micrographs, and vertical bar graphs showing differential effects for different cannabinoids in alleviating defects in differentiation and prevents hyperproliferation in psoriasis. (3A) A table outlining cannabinoid composition of Infinity (INF). (3B) Immunostaining of treated psoriatic skins (day 8) for proliferation marker Ki67 and epidermal marker Krt5. (3C-3D) Quantification of (3C) epidermal and (3D) proliferating epidermal cells after treatment. ns=no significance, **p<0.01 and ***p<0.001 compared to the control as determined by the two-tailed Student's T test. Error bars show S.E.M. n<3 in all experiments.

FIGS. 4A-4B include fluorescent micrographs and a vertical bar graph showing that INF extract, THC, and CBN attenuate NF-κB translocation. HaCaT keratinocytes were serum starved for 16 hours and then either not treated (NTC) or administered with tumor necrosis factor alpha (TNFα) along with either DMSO (Ctrl), Infinity (INF) extract, CBN, CBG, or THC. At 30 minutes post stimulation cells were harvested and stained against NF-κB and F-actin. (4A) Immunostaining of human keratinocytes stimulated with TNFα and treated with the different cannabinoids. (4B) Quantification of % nuclear NF-κB positive cells (presented in 4A). ns=no significance, ***p<0.001 and ****p<0.0001 compared to the control as determined by the two-tailed Student's T test. Error bars show S.E.M. n<3 in all experiments.

FIG. 5 includes fluorescent micrographs showing that CBN attenuates recruitment of CD3⁺ T cells (Right). IMQ-treated skins were harvested 8 days post administration of CBN or non-treated control (left), and immunostained against T cell marker CD3. Scale bars: 50 μm.

FIGS. 6A-6H include a scheme of a non-limiting study design, micrographs, vertical bar graphs and fluorescence micrographs showing that the cannabis strain Infinity (INF) attenuates acne development in vitro. (6A) Primary Blimp1⁺ cells from the mouse SG treated for 24 hours with extracts immunostained against Ki67 and F-actin. (6B) Quantification of Ki67⁺ cells (%) after treatments. (6C) Schematic non-limiting outline of acne induction via DBL: dihydrotestosterone (DHT), rosiglitazone (BRL 49653), linoleic acid (Lin), and treatment regimen in sebaceous gland (SG) organoids. (6D) SG organoids treated with INF extract. (6E) SG organoids immunostained against cleaved Caspase-3 and marker of proliferation Ki67. (6F) Quantification for percentage of proliferating cells per organoid. (6G) Quantification of organoid size after treatments. (6H) Quantification for number of cells per organoid. **p<0.01 and ***p<0.001 compared to the control as determined by two-tailed Student's t test. Error bars show S.E.M. n<3 in all experiments.

FIGS. 7A-7D include a scheme of a non-limiting study design, micrographs, vertical bar graphs and fluorescence micrographs showing that cannabinol (CBN) attenuates excessive SG cell proliferation and lipid production in DBL-induced acne in vivo. (7A) Schematic non-limiting example outline of acne induction via DBL:DHT, BRL 49653, Lin, and treatment regimen in vivo. (7B) Tail skin SGs of treated 8-week-old mice immunostained against proliferation marker Mcm2. (7C) quantification for number of proliferating SG cells after treatments. (7D) Photographs of Oil-Red-O-stained SGs. ns>0.05, *p<0.05, **p<0.01, and ***p<0.001 compared to the control as determined by two-tailed Student's t test. Error. Bars show S.E.M. n<3 in all experiments.

FIGS. 8A-8B include fluorescent micrographs and a vertical bar graph showing that INF extract, THC and CBN attenuate NF-κB translocation in sebaceous gland progenitors. (8A) Blimp1+ sebaceous gland primary progenitors were serum starved for 16 hours and then either non-treated (NTC) or administered with TNF-α along with either DMSO (Ctrl), Infinity (INF) extract, CBN, CBG, or THC. After 30 minutes post stimulation cells were harvested and stained against NF-κB antibodies and F-actin. Scale bars: 50 μm. (8B) Quantification of % nuclear NF-κB positive cells (presented in 8A). ns=no significance, *p<0.01 and ***p<0.001 compared to the control as determined by the two-tailed Student's T test.

FIGS. 9A-9J include fluorescence micrographs, a table, a scheme of a non-limiting study design, vertical bar graphs, and histological micrographs showing that cannabis strain ‘White widow’ (WW) enhances wound healing and regeneration. (9A) Immunostaining of adult Krt15-EGFP telogenic (8-week-old) dorsal skin with CB1 receptor. EGFP marks Krt15⁺ hair follicle stem cells (HFSCs). (9B) Zoom-in of HFSCs showing single channels. (9C) Table outlining cannabis strains. (9D) A non-limiting schematic outline of wound infliction and treatment regimen. (9E) Percentage of wound coverage (vs. initial wound size) of control, CNT, INF, and WW-treated wounds. (9F) Immunostaining of wound sections after 8 days post wounding with proliferation marker Ki67. (9G) Quantification of Ki67⁺ cells in the wound site at 8 days. Hematoxylin and Eosin stain of treated wound sections at 5 days PWI. (9H) Wound sections at 8 days immunostained with Krt5 and Ki67. (9I) Quantification of number of epidermal invaginations within the wound site. (9J) H&E of control, CNT, and WW-treated wound sections at 5 days PWI. *p<0.05, **p<0.01, ***p<0.001 as determined by two-tailed Student's T test. White dotted lines demarcate epidermis/dermis border. n<3 in all experiments.

FIGS. 10A-10F include a table, an image, a vertical bar graph, and fluorescence micrographs showing that the WW constituent cannabigerol (CBG) improves healing and regeneration over other cannabinoids. (10A) Table outlining cannabinoid composition of WW. (10B) Representative photograph of wounded treated mice after 8 days post wounding. (10C) Quantification of dorsal wound closure. (10D) Immunostaining of Ki67 and Krt5 in wound sections after 8 days. (10E-10F) Immunostaining of wound sections after 18 days post wound infliction with Krt15 and Ki67. ns=no significance, *p<0.05, **p<0.01, and ***p<0.001 compared to the control as determined by two-tailed Student's t test. Error bars show S.E.M. White dotted lines demarcate epidermis/dermis border. n<3 in all experiments.

FIGS. 11A-11F include micrographs, fluorescence micrographs and a vertical bar graph, showing that white widow (WW) extract and CBG drive CREB activation in vitro. and proliferation in vitro and in vivo. (11A) HaCaT keratinocytes were serum starved for 16 hours and induced with WW extract or CBG. At 30 minutes post-stimulation cells were harvested and subjected to immunofluorescence showing pCREBS133 and F-actin expression. Scale bars: 50 μm. (11B) WW extract and CBG drive CREB activation in vitro. (11C) WW extract and CBG drive keratinocyte proliferation in vitro. (11D) WW extract and CBG drive CREB activation in wounds in vivo. Neo-epidermis of wounded and treated mice after 8 days post wound infliction (PWI) showing phosphorylated CREB. CD104 marks neo-epidermal cells (e.g., basement membrane marker). (11E) quantification of pCREB+ neo-epidermal cells. Scale bars: 50 μm. ***p<0.001, *p<0.05, ns=no significance. (11F) CBG drives CREB activation in proliferating cells. Wounds were harvested and immunostained for pCREB^{s133} and Ki67 expression. Scale bars: 50 μm.

### DETAILED DESCRIPTION

The present invention provides cannabinoid compositions, plant extracts comprising cannabinoids, and methods of treating or ameliorating a disease using the described cannabinoid compounds, compositions, and extracts, in a subject in need thereof.

The present invention provides cannabinoid compositions, plant extracts comprising cannabinoids, and methods of treating or ameliorating a skin inflammatory disease using the described cannabinoid compounds, compositions, and extracts, in a subject in need thereof.

The present disclosure provides cannabinoid compositions, plant extracts comprising cannabinoids, and methods of treating or ameliorating sebaceous gland proliferative disease using the described cannabinoid compounds, compositions, and extracts, in a subject in need thereof.

The present disclosure provides cannabinoid compounds, cannabinoid compositions, plant extracts comprising cannabinoids, and methods of treating or ameliorating a wound using the described cannabinoid compounds, compositions, and extracts, in a subject in need thereof.

### Cannabinoids and Compositions

In some embodiments, the composition comprises one or more cannabinoids. In some aspects, at least one of the cannabinoids is CBN. In still other aspects, the CBN constitutes at least about 3% by weight of the total amount of cannabinoids the composition.

In some embodiments, the composition comprises one or more cannabinoids. In some aspects, at least one of the cannabinoids is CBG. In still other aspects, the CBG constitutes at least 2.5% by weight of the total amount of cannabinoids of the composition.

In some embodiments, the cannabinoid is a phytocannabinoid. As used herein, a “phytocannabinoid” is a cannabinoid that originates in nature from a Cannabis plant. Examples of cannabinoids include, but are not limited to, cannabidiol (CBD), cannabidivarin (CBDV), (−)-Δ⁹-trans-tetrahydrocannabinol (Δ⁹-THC), (−)-Δ⁹-trans-tetrahydrocannabinolic acid (Δ⁹-THCA), (−)-Δ⁹-trans-tetrahydrocannabivarin (Δ⁹-THCV), (−)-Δ⁹-trans-tetrahydrocannabivarinic acid (Δ⁹-THCVA), cannabinol (CBN), cannabivarin (CBNV), cannabicyclol (CBL), cannabigerol (CBG), cannabigerovarin (CBGV), cannabidiolic acid (CBDA), cannabichromene (CBC), cannabichromenic acid (CBCA) and any derivative thereof.

In some embodiments, the present invention is directed to a composition derived from a plant extract. In some embodiments, a plant extract of the invention is derived from a plant comprising cannabinoids. In some embodiments, the plant extract of the invention is derived from a Cannabis plant. In some embodiments, the plant extract is derived from a specific species of the Cannabis genus. In some embodiments, the cannabis plant is or comprises the “White widow” strain. In some embodiments, the cannabis plant is or comprises the “Infinity” strain. In some embodiments, the plant extract comprises any extract of a Cannabis plant comprising CBN in an amount of at least about 3% by weight of the cannabinoids of the extract.

In some embodiments, the invention relates to a composition comprising CBN (e.g., at least about 3% by weight of the cannabinoids of the composition) as an active ingredient.

In some embodiments, the disclosure relates to a composition comprising CBG (e.g., at least about 2.5% by weight of the cannabinoids of the composition) as an active ingredient.

In some embodiments, there is provided a composition comprising one to five cannabinoids substantially constituting the active agent of the composition.

In some embodiments, substantially comprises at least about 70%, at least about 80%, at least about 90%, at least about 95%, at least about 99%, or 100% by weight of the active agent of the composition, or any value and range therebetween. Each possibility represents a separate embodiment of the invention.

In some embodiments, the pharmaceutical composition comprises at least one cannabinoid. In some embodiments, the pharmaceutical composition contains at least two cannabinoids. In some embodiments, the pharmaceutical composition contains at least three cannabinoids. In still other embodiments, the pharmaceutical composition contains at least four cannabinoids. In still yet other embodiments, the pharmaceutical composition contains five cannabinoids. In still yet other embodiment, the pharmaceutical composition contains 1, 2, 3 or 4 cannabinoids.

In some embodiments, the pharmaceutical composition comprises one cannabinoid. In some embodiments, the at least one cannabinoid is CBN. In some embodiments, the at least one cannabinoid is CBG. In some embodiments, the pharmaceutical composition comprises at least two cannabinoids, wherein the cannabinoids are CBN and THC.

In some embodiments, the one to five cannabinoids comprises: CBN or CBN and THC.

In some embodiments, the at least one to five cannabinoids comprises: CBN, CBG, CBC, THC, or any combination thereof.

In some embodiments, the composition further comprises one or more additional cannabinoids. According to some embodiments, the invention relates to a composition comprising a plurality of cannabinoids.

In some embodiments, the one or more additional cannabinoids is selected from: CBG, CBC, CBGA, CBG-C4, CBCA, CBC-C4, CBD, CBDV, CBDA, CBDVA, CBGV, CBGM, SesquiCBG, THC (including Δ⁸ THC, and/or Δ⁹ THC), THCA, THCV (including Δ⁹ THCV), THCVA (including Δ⁹ THCVA) CBDA, CBDA-C4, CBD-C4, CBDVA, CBDO, CBDM, CBCVA, CBCMA, CBCV, CBCO, CBN, CBNV, OH—CBN, OH-CBNA, CBEA, CBE, CBEV, CBEVA, CBDVA, CBNDA, CBND, CBL, CBT-1, CBTV-1, CBT-3, CBT-2, or any combination thereof. Each possibility represents a separate embodiment of the invention.

In some embodiments, THC is or comprises Δ⁸-THC, Δ⁹-THC, or both.

In some embodiments, THCV is or comprises Δ⁹-THCV.

In some embodiments, there is provided a composition comprising CBN or CBN and THC. In some embodiments, the composition consists essentially of CBN. In some embodiments, the composition consists essentially of CBN and THC.

In some embodiments, the pharmaceutical composition comprises CBN and at least one additional cannabinoid. In some embodiments, the composition comprises CBN and CBG. In some embodiments, the composition comprises CBN and CBC. In some embodiments, the composition comprises CBN and THC. In some embodiments, the composition comprises CBN, CBG, CBC, and THC.

In some embodiments, the pharmaceutical composition comprises THC and at least one additional cannabinoid. In some embodiments, the composition comprises THC and CBN. In some embodiments, the composition comprises THC and CBC. In some embodiments, the composition comprises THC and CBG.

In some embodiments, there is provided a composition comprising CBG and THC.

In some embodiments, the composition is a pharmaceutical composition.

In some embodiments, at least about 0.1%, 0.5%, 1%, 2%, 3%, 5%, 10%, 20%, 30%, 50%, 70%, 85%, 90%, 99% or 100% of the cannabinoid content of the composition is CBN, or any value and range therebetween. Each possibility represents a separate embodiment of the invention. In some embodiments, the composition comprises at most about 0.5%, 1%, 5%, 10%, 25%, 40%, 50%, 60%, 70%, 80%, 90%, 95% or 99% CBN, or any value and range therebetween. Each possibility represents a separate embodiment of the invention.

In some embodiments, at least about 1%, 5%, 10%, 20%, 30%, 40%, 50%, 60%, 70%, 80%, 90%, or 95%, of the cannabinoid content of the composition is THC, or any value and range therebetween. Each possibility represents a separate embodiment of the invention. In some embodiments, the composition comprises at most about 1%, 5%, 10%, 20%, 30%, 40%, 50%, 60%, 70%, 80%, 90%, 95% or 97% THC, or any value and range therebetween. Each possibility represents a separate embodiment of the invention. Each possibility represents a separate embodiment of the invention.

In some embodiments, at least 0.1%, 0.5%, 1%, 2%, 3%, 5%, 10%, 20%, 30%, 50%, 70%, 85%, 90%, 99% or 100% of the cannabinoid content of the composition is CBG, or any value and range therebetween. Each possibility represents a separate embodiment of the disclosure. In some embodiments, the composition comprises at most 0.5%, 1%, 5%, 10%, 25%, 40%, 50%, 60%, 70%, 80%, 90%, 95% or 99% CBG, or any value and range therebetween. Each possibility represents a separate embodiment of the disclosure.

In some embodiments, at least 1%, 5%, 10%, 20%, 30%, 40%, 50%, 60%, 70%, 80%, 90%, or 95% of the cannabinoid content of the composition is CBC, or any value and range therebetween. Each possibility represents a separate embodiment of the disclosure. In some embodiments, the composition comprises at most 1%, 5%, 10%, 20%, 30%, 40%, 50%, 60%, 70%, 80%, 90%, or 95% CBC, or any value and range therebetween. Each possibility represents a separate embodiment of the disclosure. Each possibility represents a separate embodiment of the disclosure.

In some embodiments, CBN and THC combined, comprise at least about 10%, 15%, 25%, 30%, 45%, 50%, 60%, 70%, 80%, 85%, 90%, 97%, or 99% by weight, of the total cannabinoids of composition, or any value and range therebetween. Each possibility represents a separate embodiment of the invention. In some embodiments, CBN and THC combined, comprise at least about 5-20%, 10-35%, 20-35%, 45-80%, 50-75%, 60-95%, 70-99%, 80-100%, 50-85%, 60-90%, 68-97%, or 55-99% by weight, of the total cannabinoids of composition. Each possibility represents a separate embodiment of the invention.

In some embodiments, CBN and THC, or any combination thereof, constitutes more than 50% by weight of the cannabinoids of the composition.

In some embodiments, the composition comprises a w/w ratio of (i) CBN and (ii) THC, selected from about 1:5 to 1:25; 1:10 to 1:20; 1:20 to 1:40; 1:10 to 1:40, or 1:15 to 1:35, wherein each possibility represents a separate embodiment of the invention.

In some embodiments, CBN, CBG, CBC, and THC, combined, comprise at least 10%, 15%, 25%, 30%, 45%, 50%, 60%, 70%, 80%, 85%, 90%, 97%, or 99% by weight, of the total cannabinoid composition, or any value and range therebetween. Each possibility represents a separate embodiment of the disclosure. In some embodiments, CBN, CBG, CBC, and THC, combined, comprise at least 5-20%, 10-35%, 20-35%, 45-80%, 50-75%, 60-95%, 70-99%, 80-100%, 50-85%, 60-90%, 68-97%, or 55-99% by weight, of the total cannabinoids of composition. Each possibility represents a separate embodiment of the disclosure.

In some embodiments, CBN, CBG, CBC, THC, or any combination thereof, constitutes more than 50% by weight of the cannabinoids of the composition.

In some embodiments, the composition comprises a w/w ratio of (i) CBN and (ii) at least one of CBG, CBC, or any combination thereof, selected from 2:1 to 1:6; 1:1 to 1:5; 1:1 to 1:4; 2:1 to 1:5; 2:1 to 1:3; or 1:1 to 1:2, wherein each possibility represents a separate embodiment of the disclosure.

In some embodiments, the composition comprises a w/w ratio of (i) CBN and (ii) CBG, selected from 4:1 to 1:1; 3:1 to 1:2; or 1:1 to 1:4; or 2:1 to 1:2, wherein each possibility represents a separate embodiment of the disclosure.

In some embodiments, the composition comprises a w/w ratio of (i) CBN and (ii) CBC, selected from 5:1 to 1:0.1; 5:1 to 1:1; 4:1 to 1:1; 3:1 to 1:1; 2:1 to 1:1; or 3:1 to 1:0.1, wherein each possibility represents a separate embodiment of the disclosure.

In some embodiments, the composition comprises a w/w ratio of (i) CBG and (ii) CBC, selected from 4:1 to 1:1; 4:1 to 2:1; 3:1 to 2:1; 3:1 to 1:1; 4:1 to 1:0.1; or 3:1 to 1:0.1, wherein each possibility represents a separate embodiment of the disclosure.

In some embodiments, the composition comprises a w/w ratio of (i) CBG and (ii) THC, selected from 1:5 to 1:35; 1:10 to 1:40; 1:30 to 1:35; 1:15 to 1:45; 1:20 to 1:50; or 1:10 to 1:60, wherein each possibility represents a separate embodiment of the disclosure.

In some embodiments, the composition is for use in the treatment of sebaceous gland proliferative disease.

In some embodiments, the cannabinoid is not a psychoactive cannabinoid.

In some embodiments, CBG and THC combined, comprise at least 10%, 15%, 25%, 30%, 45%, 50%, 60%, 70%, 80%, 85%, 90%, 97%, or 99% by weight, of the total cannabinoids of composition, or any value and range therebetween. Each possibility represents a separate embodiment of the disclosure. In some embodiments, CBG and THC combined, comprise at least 5-20%, 10-35%, 20-35%, 45-80%, 50-75%, 60-95%, 70-99%, 80-100%, 50-85%, 60-90%, 68-97%, or 55-99% by weight, of the total cannabinoids of composition. Each possibility represents a separate embodiment of the disclosure.

In some embodiments, CBG and THC, or any combination thereof, constitutes more than 50% by weight of the cannabinoids of the composition.

In some embodiments, the composition comprises a w/w ratio of (i) CBG and (ii) THC, selected from 1:5 to 1:25; 1:10 to 1:20; 1:20 to 1:40; 1:10 to 1:40, or 1:15 to 1:35, wherein each possibility represents a separate embodiment of the disclosure.

In some embodiments, the pharmaceutical composition comprising at least one active agent and at least one pharmaceutically acceptable excipient, wherein the active agent comprises from one to five cannabinoids and further wherein at least one of the cannabinoids is cannabinol (CBN).

In some embodiments, the pharmaceutical composition for treating a sebaceous gland proliferative disease, comprising at least one active agent and at least one pharmaceutically acceptable excipient, wherein the active agent comprises from one to five cannabinoids and further wherein at least one of the cannabinoids is cannabinol (CBN).

In some embodiments, the composition is for use in the treatment of a wound (also referred to herein as “wound healing”). In some embodiments, treatment of a wound according to the herein disclosed method comprises induction of cell proliferation in a wound site. In some embodiments, treatment of a wound according to the herein disclosed method comprises induction of epithelial cell proliferation in a wound site. In some embodiments, the composition is for use in the treatment of alopecia.

In some embodiments, the pharmaceutical composition for treating a wound, to regenerate skin, or for hair follicle regeneration, comprising at least one active agent and at least one pharmaceutically acceptable excipient, wherein the active agent comprises from one to five cannabinoids and further wherein at least one of the cannabinoids is cannabigerol (CBG).

In some embodiments, the active agent of the pharmaceutical composition comprises at least two cannabinoids, at least three cannabinoids, at least four cannabinoids or five cannabinoids.

In some embodiments, the active agent of the pharmaceutical composition comprises at least two cannabinoids, at least three cannabinoids, at least four cannabinoids or five cannabinoids.

In some embodiments, the pharmaceutical composition comprises at least about 2.0%, 2.5%, or 3% by weight of the one to five cannabinoids comprises CBN, or any value and range therebetween. Each possibility represents a separate embodiment of the invention.

In some embodiments, at least about 2.0%, 2.2%, or 2.5% by weight of the one to five cannabinoids comprises THC, or any value and range therebetween. Each possibility represents a separate embodiment of the invention.

In some embodiments, at least about 2.0%, 2.2%, or 2.5% by weight of the one to five cannabinoids comprises CBG, or any value and range therebetween. Each possibility represents a separate embodiment of the invention.

In some embodiments, the composition further comprises tetrahydrocannabinol (THC).

In some embodiments, the composition further comprises one or more cannabinoids elected from tetrahydrocannabinol (THC), cannabigerol (CBG), or cannabichromene (CBC).

In some embodiments, the pharmaceutical composition further comprises tetrahydrocannabinol (THC) wherein the THC is (−)-Δ⁹-trans-tetrahydrocannabinol (Δ⁹-THC).

In some embodiments, the active agent of the pharmaceutical composition further comprises at least one of: cannabidiol (CBD), cannabidivarin (CBDV), (−)-Δ⁹-trans-tetrahydrocannabinolic acid (Δ⁹-THCA), (−)-Δ⁹-trans-tetrahydrocannabivarin (Δ⁹-THCV), (−)-Δ⁹-trans-tetrahydrocannabivarinic acid (Δ⁹-THCVA), cannabivarin (CBNV), cannabicyclol (CBL), cannabigerovarin (CBGV), cannabidiolic acid (CBDA), cannabichromenic acid (CBCA), or any combination thereof.

In some embodiments, active agent comprises at least about 80% by weight of the pharmaceutical composition.

In some embodiments, the active agent comprises CBN and THC in a ratio of about 1:10 to about 1:40 (w/w).

In some embodiments, the active agent comprises CBG and THC in a ratio of about 1:10 (w/w) to 1:35 (w/w).

In some embodiments, the active agent does not contain cannabinol (CBN) or cannabichromene (CBC).

In some embodiments, at least one of the one to five cannabinoids comprises a highly purified extract of Cannabis.

In some embodiments, at least one of the one to five cannabinoids comprises a synthetically produced cannabinoid.

In some embodiments, the active agent does not contain any cannabigerol (CBG) or cannabichromene (CBC), or both.

In some embodiments, the active agent comprises CBN and CBG in a ratio of about 2:1 to about 1:2 (w/w).

In some embodiments, the active agent comprises CBN and CBC in a ratio of about 5:1 to about 1:1 (w/w).

In some embodiments, the pharmaceutical composition is a tablet, a capsule, a gel, a semi-liquid, a liquid, a solution, an emulsion, a spray, a chewable food product, or any combination thereof.

In some embodiments, there is provided a method for treating a subject suffering from an inflammatory skin disease, the method comprising administering to the subject in need thereof a therapeutically effective amount of a composition comprising at least one active agent and at least one pharmaceutically acceptable excipient, wherein the active agent comprises from one to five cannabinoids and further wherein at least one of the cannabinoids is cannabinol (CBN).

In some embodiments, the inflammatory skin disease is a keratinocyte-related inflammatory disease.

In some embodiments, the keratinocyte-related inflammatory disease is psoriasis, allergic contact dermatitis, atopic dermatitis, or any combination thereof.

In some embodiments, it is provided a method for treating a subject with a wound, for regenerating skin, or for hair follicle regeneration, the method comprising administering to the subject in need thereof a therapeutically effective amount of the composition comprising at least one active agent and at least one pharmaceutically acceptable excipient, wherein the active agent comprises from one to five cannabinoids and further wherein at least one of the cannabinoids is cannabigerol (CBG).

In some embodiments, the method of treating comprises reducing: the area size of the wound, increasing the number of skin invaginations, increasing the number of skin proliferating cells, increasing the proliferation rate of skin cells, inducing hair follicle growth or regeneration, inhibiting, or reducing scarring, or any combination thereof, in the subject.

In some embodiments, it is provided a method for treating a subject with a sebaceous gland proliferative disease, the method comprising administering to the subject in need thereof a therapeutically effective amount of the composition comprising at least one active agent and at least one pharmaceutically acceptable excipient, wherein the active agent comprises from one to five cannabinoids and further wherein at least one of the cannabinoids is cannabinol (CBN).

In some embodiments, the method of treating comprises reducing: the number, rate, or both, of proliferating sebaceous cells, the size, growth rate, or both of sebaceous glands, sebum amount, production rate, or both, or any combination thereof, in the subject.

In some embodiments, the sebaceous gland proliferative disease is a hyperproliferative disease.

In some embodiments, the sebaceous gland proliferative disease is selected from the group consisting of: acne, sebaceous carcinoma, sebaceous hyperplasia, seborrhoea, seborrhoeic dermatitis, sebopsoriasis, sebaceous adenoma, nevus sebaceous, or phymatous rosacea.

In some embodiments, the subject is a human subject.

As used herein, the term “plurality of cannabinoids” refers to two or more cannabinoids, e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, and at least 30, or any value and range therebetween. Each possibility represents a separate embodiment of the invention. In some embodiments, the plurality of cannabinoids of the composition are those having a relative amount of at least about 2%, at least about 1.5%, at least about 1%, at least about 0.4%, at least about 0.3%, at least about 0.2%, at least about 0.1%, or any value and range therebetween. Each possibility represents a separate embodiment of the invention.

In some embodiments, the composition of the invention comprises at least about 0.01%, 0.1%, 0.5%, 1%, 5%, 10%, 20%, 30%, 40%, 50%, 60%, 70%, 80%, 90%, 95%, 99% cannabinoids, or any value and range therebetween. Each possibility represents a separate embodiment of the invention. In some embodiments, the plant extract of the invention comprises at most about 0.01%, 0.1%, 0.5%, 1%, 5%, 10%, 20%, 30%, 40%, 50%, 60%, 70%, 80%, 90%, 95%, 99% or 100% cannabinoids, or any value and range therebetween. Each possibility represents a separate embodiment of the invention. In some embodiments, the composition comprises at least about 0.001%, 0.01%, 0.1%, 1%, 5%, 10%, 20%, 30%, 40%, 50%, 60%, 70%, 80%, 90%, 95%, 99% CBN, or any value and range therebetween. Each possibility represents a separate embodiment of the invention. In some embodiments, the composition comprises at most about 0.1%, 0.5%, 1%, 5%, 10%, 20%, 30%, 40%, 50%, 60%, 70%, 80%, 90%, 95% or 99% CBN, or any value and range therebetween. Each possibility represents a separate embodiment of the invention. In some embodiments, the composition comprises at least about 0.001%, 0.01%, 0.1%, 1%, 5%, 10%, 20%, 30%, 40%, 50%, 60%, 70%, 80%, 90%, 95%, 99% CBN and THC, or any value and range therebetween. Each possibility represents a separate embodiment of the invention. In some embodiments, the composition comprises at most about 0.1%, 0.5%, 1%, 5%, 10%, 20%, 30%, 40%, 50%, 60%, 70%, 80%, 90%, 95% or 99% CBN and THC, or any value and range therebetween. Each possibility represents a separate embodiment of the invention.

In some embodiments, the composition comprises at least 0.001%, 0.01%, 0.1%, 1%, 5%, 10%, 20%, 30%, 40%, 50%, 60%, 70%, 80%, 90%, 95%, 99% CBC, or any value and range therebetween. Each possibility represents a separate embodiment of the disclosure. In some embodiments, the composition comprises at most 0.1%, 0.5%, 1%, 5%, 10%, 20%, 30%, 40%, 50%, 60%, 70%, 80%, 90%, 95% or 99% CBC, or any value and range therebetween. Each possibility represents a separate embodiment of the disclosure. In some embodiments, the composition comprises at least 0.001%, 0.01%, 0.1%, 1%, 5%, 10%, 20%, 30%, 40%, 50%, 60%, 70%, 80%, 90%, 95%, 99% CBG, or any value and range therebetween. Each possibility represents a separate embodiment of the disclosure. In some embodiments, the composition comprises at most 0.1%, 0.5%, 1%, 5%, 10%, 20%, 30%, 40%, 50%, 60%, 70%, 80%, 90%, 95% or 99% CBG, or any value and range therebetween. Each possibility represents a separate embodiment of the disclosure. In some embodiments, the composition comprises at least 0.001%, 0.01%, 0.1%, 1%, 5%, 10%, 20%, 30%, 40%, 50%, 60%, 70%, 80%, 90%, 95%, 99% THC, or any value and range therebetween. Each possibility represents a separate embodiment of the disclosure. In some embodiments, the composition comprises at most 0.1%, 0.5%, 1%, 5%, 10%, 20%, 30%, 40%, 50%, 60%, 70%, 80%, 90%, 95% or 99% THC, or any value and range therebetween. Each possibility represents a separate embodiment of the disclosure.

In some embodiments, the composition of the disclosure comprises at least 0.01%, 0.1%, 0.5%, 1%, 5%, 10%, 20%, 30%, 40%, 50%, 60%, 70%, 80%, 90%, 95%, 99% cannabinoids, or any value and range therebetween. Each possibility represents a separate embodiment of the disclosure. In some embodiments, the plant extract of the disclosure comprises at most 0.01%, 0.1%, 0.5%, 1%, 5%, 10%, 20%, 30%, 40%, 50%, 60%, 70%, 80%, 90%, 95%, 99% or 100% cannabinoids, or any value and range therebetween. Each possibility represents a separate embodiment of the disclosure. In some embodiments, the composition comprises at least 0.001%, 0.01%, 0.1%, 1%, 5%, 10%, 20%, 30%, 40%, 50%, 60%, 70%, 80%, 90%, 95%, 99% CBG, or any value and range therebetween. Each possibility represents a separate embodiment of the disclosure. In some embodiments, the composition comprises at most 0.1%, 0.5%, 1%, 5%, 10%, 20%, 30%, 40%, 50%, 60%, 70%, 80%, 90%, 95% or 99% CBG, or any value and range therebetween. Each possibility represents a separate embodiment of the disclosure.

In some embodiments, the composition comprises or consists of a plant extract.

As used herein, the term “extract” comprises the whole extract, a fraction thereof, a portion thereof, an isolated compound therefrom, or any combination thereof.

In some embodiments, the extract is derived from a plant material.

In some embodiments, the plant material is first dried and then extracted. In some embodiments, the plant material is air-dried. In some embodiments, the plant material is further heat treated (e.g., hot-drying) and then extracted.

As used herein, treatment before extraction comprises, for example, freezing, drying, lyophilizing, or any combination thereof.

In some embodiments, the plant material is further processed prior to the extraction procedure in order to facilitate the extraction procedure. In some embodiments, processing methods prior to extraction, include but are not limited to crushing, slicing, or shredding, such as by using a grinder or other devices to fragment the plant parts into small pieces or powder.

In some embodiments, the extraction is a solvent-based extraction. In some embodiments, the solvent is a polar solvent. As used herein, a polar solvent may be selected from the group including, but not limited to, ethanol and Isopropyl. In some embodiments, the solvent is a non-polar solvent. In some embodiments, the extraction is a solvent-less-based extraction.

According to some embodiments, there is provided a pharmaceutical composition comprising the herein disclosed cannabinoids and a pharmaceutically acceptable carrier.

In some embodiments, the Cannabis derived substance used in the composition and methods as described herein includes CBN. In one embodiment, the composition described herein comprises purified or substantially purified (e.g., greater than 80% w/w, 85% w/w, 90%, w/w 95% w/w or 97% w/w) CBN. In some embodiments of the methods described herein, purified, or substantially purified (e.g., greater than 80% w/w, 85% w/w, 90%, w/w 95% w/w or 97% w/w) CBN is administered to a subject suffering from a skin inflammatory disease as described herein.

In one embodiment, the Cannabis derived substances used in the composition and methods as described herein include CBN and THC. In one embodiment, the composition described herein comprises purified or substantially purified (e.g., greater than about 80% w/w, about 85% w/w, about 90% w/w, about 95% w/w, or about 97% w/w) CBN and THC. In some embodiments of the methods described herein, purified, or substantially purified (e.g., greater than about 80% w/w, about 85% w/w, about 90% w/w, about 95% w/w, or about 97% w/w) CBN and THC, is administered to a subject suffering from a skin inflammatory disease.

In one embodiment, the Cannabis derived substances used in the composition and methods as described herein include CBC, or a functional variant thereof. In one embodiment, the composition described herein comprises purified or substantially purified (e.g., greater than 80% w/w, 85% w/w, 90%, w/w 95% w/w or 97% w/w) CBC. In some embodiments of the methods described herein, purified or substantially purified (e.g., greater than 80% w/w, 85% w/w, 90%, w/w 95% w/w or 97% w/w) CBC, or a functional variant thereof, is administered to a subject suffering from a sebaceous gland proliferative disease.

In some embodiments, the Cannabis derived substance used in the composition and methods as described herein includes CBG. In one embodiment, the composition described herein comprises purified or substantially purified (e.g., greater than 80% w/w, 85% w/w, 90%, w/w 95% w/w or 97% w/w) CBG. In some embodiments of the methods described herein, purified or substantially purified (e.g., greater than 80% w/w, 85% w/w, 90%, w/w 95% w/w or 97% w/w) CBG is administered to a subject suffering from a wound.

In one embodiment, the Cannabis derived substances used in the composition and methods as described herein include THC, or a functional variant thereof. In one embodiment, the composition described herein comprises purified or substantially purified (e.g., greater than 80% w/w, 85% w/w, 90%, w/w 95% w/w or 97% w/w) THC. In some embodiments of the methods described herein, purified or substantially purified (e.g., greater than 80% w/w, 85% w/w, 90%, w/w 95% w/w or 97% w/w) THC, or a functional variant thereof, is administered to a subject suffering from a wound.

As used herein, the term “synthetic cannabinoids” refers to compounds that have a cannabinoid or cannabinoid-like structure and are manufactured using chemical means rather than by the plant.

As used herein, the term “carrier”, “excipient” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

A pharmaceutical composition may take any physical form necessary for proper administration. The composition comprising an encapsulated one or more cannabinoid compounds can be administered in any suitable form, including but not limited to a liquid form, a gel form, a semi-liquid (e.g., a liquid, such as a viscous liquid, containing some solid) form, a semi-solid (a solid containing some liquid) form, or a solid form. Compositions can be provided in, for example, a tablet form, a capsule form, a liquid form, a food form a chewable form, a non-chewable form, a transbuccal form, a sublingual form, a slow-release form, a non-slow-release form, a sustained release form, or a non-sustained-release form.

A pharmaceutically-acceptable carrier suitable for the preparation of unit dosage form of a composition as described herein for peroral administration is well-known in the art.

In some embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmellose sodium, crospovidone, guar gum, sodium starch glycolate), additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), stabilizers (e.g. hydroxypropyl cellulose, hydroxypropylmethyl cellulose), viscosity increasing agents (e.g. carbomer, colloidal silicon dioxide, ethyl cellulose, guar gum lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), polymer coatings (e.g., poloxamers or poloxamines), and/or coating and film forming agents (e.g. ethyl cellulose, acrylates, polymethacrylates).

In some embodiments, preparation of effective amount or dose can be estimated initially from in vitro assays. In one embodiment, a dose can be formulated in animal models, and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

### Methods of Treatment/Use

According to Some Embodiments, there is Provided a Method for Treating a Subject afflicted with a keratinocyte-related inflammatory disease, comprising administering to the subject a therapeutically effective amount of a pharmaceutical composition comprising CBN or CBN and THC.

As used herein, the term “keratinocyte-related inflammatory disease” refers to any disease or disorder involving inflammatory response of keratinocytes as part of the disease or disorder pathogenesis or pathophysiology.

In some embodiments, the keratinocytes-related inflammatory disease is selected from the group consisting of: psoriasis, allergic contact dermatitis, atopic dermatitis, seborrheic dermatitis, acne, blisters, rosacea, hives, actinic keratosis, Cellulitis, measles, vitiligo, warts, keratosis pilaris, keratodermas, impetigo, and hidradenitis suppurativa.

In some embodiments, the herein disclosed method is directed to treating a subject afflicted with a skin inflammatory disease.

In some embodiments, treating comprises inhibiting or reducing NF-κB migration from the cytosol to the nucleus, inhibiting or reducing NF-κB signaling, % of nuclear NF-κB, or any combination thereof, in keratinocytes of a subject.

Methods for determining NF-κB migration from the cytosol to the nucleus, NF-κB signaling, or % of nuclear NF-κB, are common and would be apparent to one of ordinary skill in the art. Non-limiting examples for such methods include, but are not limited to PCR, RT-PCR, western blot, microscopy, immunohistochemistry, or other, such as exemplified herein.

In some embodiments, a skin inflammatory disease comprises or is a keratinocytes-related inflammatory disease.

According to some embodiments, there is provided a method for treating a subject afflicted with a keratinocytes-related inflammatory disease, comprising administering to the subject a therapeutically effective amount of a pharmaceutical composition comprising CBN or CBN and THC.

According to some embodiments, there is provided a method for treating a subject afflicted with a skin inflammatory disease in a subject in need thereof, comprising administering to the subject a therapeutically effective amount of a pharmaceutical composition comprising one to five cannabinoids substantially constituting the active agent of the composition.

According to some embodiments, there is provided a method for treating a subject afflicted with a keratinocytes-related inflammatory disease comprising administering to the subject a therapeutically effective amount of a composition comprising CBN or CBN and THC as the active agent, thereby treating a subject afflicted with a keratinocytes-related inflammatory disease.

According to some embodiments, the method comprises treating or ameliorating inflammation or a symptom associated therewith.

As used herein, the terms “administering”, “administration”, and like terms refer to any method which, in sound medical practice, delivers a composition containing an active agent to a subject in such a manner as to provide a therapeutic effect. One aspect of the present subject matter provides for dermal or transdermal administration of a therapeutically effective amount of a composition of the present subject matter to a subject in need thereof. Other suitable routes of administration can include oral, dermal, transdermal, parenteral, subcutaneous, intravenous, intramuscular, or intraperitoneal. In some embodiments, the administering is systemic administering. In some embodiments, the administering is to the inflamed skin site.

In some embodiments, there is provided a composition for use in the treatment of a keratinocytes-related inflammatory disease. In some embodiments, treatment of a keratinocytes-related inflammatory disease according to the herein disclosed method comprises inhibiting or reducing NF-κB migration from the cytosol to the nucleus, inhibiting or reducing NF-κB signaling, % of nuclear NF-κB, or any combination thereof, in keratinocytes of a subject.

In some embodiments, the keratinocytes induce an inflammatory response in the subject. In some embodiments, the keratinocytes enhance, promote, or propagate an inflammatory response in the subject.

It would be apparent to a person of skill in the art that keratinocytes are keratinocytes of the skin.

In some embodiments, compositions for use in the methods of this invention comprise solutions or emulsions, which in some embodiments are aqueous solutions or emulsions comprising a safe and effective amount of the cannabinoids of the present invention and optionally, other compounds as described herein, including excipients intended for topical intranasal administration.

Administering the composition to a specific site in the subject may be performed with any method known in the art. This may include with an applicator, in the form of a gel or cream, as well as on a scaffold, wrap or bandage.

As used herein, the terms “treatment” or “treating” of a disease, disorder or condition (e.g., inflammation) encompasses alleviation of at least one symptom thereof, a reduction in the severity thereof, or inhibition of the progression thereof. Treatment need not mean that the disease, disorder or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, “treating” comprises ameliorating and/or preventing.

In some embodiments, the composition of the invention inhibits or reduces the percentage (%) of nuclear NF-κB positive cells or keratinocytes.

In some embodiments, inhibits or reduces is at least 5%, 10%, 35%, 50%, 80%, 100%, 150%, 270%, 400%, 650%, 800%, or 1,000% inhibition or reduction compared to a control or any value and range therebetween. Each possibility represents a separate embodiment of the invention.

In some embodiments, a control is a non-treated subject. In some embodiments, a control is a non-active cannabinoid. In some embodiments, a non-active cannabinoid is not active in the context of inducing NF-κB migration to the nucleus.

According to some embodiments, there is provided a method for treating a subject afflicted with a sebaceous gland proliferative disease, comprising administering to the subject a therapeutically effective amount of a pharmaceutical composition comprising one or more cannabinoids. According to some embodiments, the pharmaceutical composition comprises CBN and additional cannabinoids. According to some embodiments, the pharmaceutical composition comprises THC and additional cannabinoids. According to some embodiments, the pharmaceutical composition comprises CBN and CBG. According to some embodiments, the pharmaceutical composition comprises CBN and CBC. According to some embodiments, the pharmaceutical composition comprises CBN and THC. According to some embodiments, the pharmaceutical composition comprises CBN, CBG, and THC. According to some embodiments, the pharmaceutical composition comprises CBN, CBC, and THC. According to some embodiments, the pharmaceutical composition comprises CBN, CBG, CBC, and THC.

According to some embodiments, there is provided a method for treating a subject afflicted with a sebaceous gland proliferative disease, comprising administering to the subject a therapeutically effective amount of a pharmaceutical composition comprising one or more cannabinoids substantially constituting the active agent of the composition.

In some embodiments, one or more comprises one to five.

According to some embodiments, there is provided a method for treating a subject in need of inhibition or reduction of sebaceous gland cell proliferation, comprising administering to the subject a therapeutically effective amount of a pharmaceutical composition comprising one or more cannabinoids. According to some embodiments, the pharmaceutical composition comprises CBN. According to some embodiments, the pharmaceutical composition comprises CBN and CBG. According to some embodiments, the pharmaceutical composition comprises CBN and CBC. According to some embodiments, the pharmaceutical composition comprises CBN and THC. According to some embodiments, the pharmaceutical composition comprises CBN, CBG, and THC. According to some embodiments, the pharmaceutical composition comprises CBN, CBC, and THC. According to some embodiments, the pharmaceutical composition comprises CBN, CBG, CBC, and THC.

As used herein, the term “sebaceous gland proliferative disease” encompasses any disease involving and/or resulting in abnormally regulated, dysregulated, upregulated, or any combination thereof, proliferation of at least one sebaceous gland cell.

In some embodiments, a sebaceous gland proliferative disease is characterized by hypertrophic sebaceous gland, hyperplastic sebaceous gland, or both.

In some embodiments, a sebaceous gland proliferative disease is selected from: acne, sebaceous carcinoma, sebaceous hyperplasia, seborrhoea, seborrhoeic dermatitis, sebopsoriasis (also known as “seborrhiasis”), sebaceous adenoma, nevus sebaceous, or phymatous rosacea.

According to some embodiments, there is provided a method for treating a subject afflicted with a sebaceous gland proliferative disease, comprising administering to the subject a therapeutically effective amount of a composition comprising at least one cannabinoid as the active agent, thereby treating a subject afflicted with a sebaceous gland proliferative disease. According to some embodiments, the pharmaceutical composition comprises CBN as the active agent, thereby treating a subject afflicted with a sebaceous gland proliferative disease. According to some embodiments, the pharmaceutical composition comprises THC as the active agent, thereby treating a subject afflicted with a sebaceous gland proliferative disease. According to some embodiments, the pharmaceutical composition comprises CBN and CBG as the active agent, thereby treating a subject afflicted with a sebaceous gland proliferative disease. According to some embodiments, the pharmaceutical composition comprises CBN and CBC as the active agent, thereby treating a subject afflicted with a sebaceous gland proliferative disease. According to some embodiments, the pharmaceutical composition comprises CBN and THC as the active agent, thereby treating a subject afflicted with a sebaceous gland proliferative disease. According to some embodiments, the pharmaceutical composition comprises CBN, CBG, and THC as the active agent, thereby treating a subject afflicted with a sebaceous gland proliferative disease. According to some embodiments, the pharmaceutical composition comprises CBN, CBC, and THC as the active agent, thereby treating a subject afflicted with a sebaceous gland proliferative disease. According to some embodiments, the pharmaceutical composition comprises CBN, CBG, CBC, and THC as the active agent, thereby treating a subject afflicted with a sebaceous gland proliferative disease.

According to some embodiments, the pharmaceutical composition comprises THC and CBG as the active agent, thereby treating a subject afflicted with a sebaceous gland proliferative disease. According to some embodiments, the pharmaceutical composition comprises THC and CBC as the active agent, thereby treating a subject afflicted with a sebaceous gland proliferative disease. According to some embodiments, the pharmaceutical composition comprises THC and CBN as the active agent, thereby treating a subject afflicted with a sebaceous gland proliferative disease. According to some embodiments, the pharmaceutical composition comprises THC, CBN, and CBG, as the active agent, thereby treating a subject afflicted with a sebaceous gland proliferative disease. According to some embodiments, the pharmaceutical composition comprises THC, CBN, and CBC, as the active agent, thereby treating a subject afflicted with a sebaceous gland proliferative disease.

According to some embodiments, the method comprises treating or preventing a sebaceous gland proliferative disease.

In some embodiments, the method comprises or is directed to ameliorating at least one symptom in the subject afflicted with a sebaceous gland proliferative disease.

In some embodiments, the administering is to the site of sebaceous gland proliferation.

Administering the composition to a specific site in the subject may be performed with any method known in the art. This may include with an applicator, in the form of a gel or cream, as well as on a scaffold, wrap or bandage.

As used herein, the terms “treatment” or “treating” of a disease, disorder or condition encompasses alleviation of at least one symptom thereof, a reduction in the severity thereof, or inhibition of the progression thereof. Treatment need not mean that the disease, disorder or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, alleviated symptoms of the disease, disorder or condition include reduced sebaceous gland (SG) cell viability, inhibited or reduced SG cell proliferation including proliferation rate, reduced sebaceous gland length, size, or both.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “proliferative disease” comprises a disease or disorder characterized by an increase of cell proliferation. In some embodiments, a subject is characterized by comprising an increased number of proliferating cells. In some embodiments, the cell proliferation is an abnormal cell proliferation. In some embodiments, the cell proliferation is an unregulated or dysregulated cell proliferation.

In some embodiments, the composition of the disclosure reduces the viability of a sebaceous gland (SG) cell. In some embodiments, the composition induces apoptosis of a SG cell. In some embodiments, the composition reduces the proliferation or rate thereof of a SG cell.

In some embodiments, the proliferation rate of a SG cell contacted with the composition of the disclosure is reduced or inhibited by at least 5%, 10%, 15%, 20%, 30%, 40%, 50%, 60%, 70%, 80%, 90% or 100% compared to a control cell, or any value and range therebetween. Each possibility represents a separate embodiment of the disclosure.

According to some embodiments, there is provided a method for treating a subject afflicted with a wound, comprising administering to the subject a therapeutically effective amount of a pharmaceutical composition comprising one or more cannabinoids. According to some embodiments, the pharmaceutical composition comprises CBG and additional cannabinoids. According to some embodiments, the pharmaceutical comprises CBG and THC.

According to some embodiments, there is provided a method for treating a wound, regenerating skin, or both, in a subject in need thereof, comprising administering to the subject a therapeutically effective amount of a pharmaceutical composition comprising one to five cannabinoids substantially constituting the active agent of the composition.

According to some embodiments, there is provided a method for treating a subject in need of induction of cell proliferation, comprising administering to the subject a therapeutically effective amount of a pharmaceutical composition comprising one or more cannabinoids. According to some embodiments, the pharmaceutical composition comprises CBG. According to some embodiments, the pharmaceutical composition comprises CBG and THC.

In some embodiments, the subject is afflicted with a wound. In some embodiments, the subject is in need of induction of epithelial cell proliferation. In some embodiments, the subject is afflicted with alopecia.

Methods for determining or diagnosing alopecia in a subject are common and would be apparent to a skilled physician. Non limiting example for such a method, may include, but is not limited to, biopsy analysis. Blood tests may also be employed so as to rule out an autoimmune disease or disorder.

According to some embodiments, there is provided a method for treating a subject afflicted with a wound, comprising administering to the subject a therapeutically effective amount of a composition comprising at least one cannabinoid as the active agent, thereby treating a subject afflicted with a wound. According to some embodiments, the pharmaceutical composition comprises CBG as the active agent, thereby treating a subject afflicted with a wound. According to some embodiments, the pharmaceutical composition comprises CBG and THC as the active agent, thereby treating a subject afflicted with a wound.

According to some embodiments, the method comprises treating or ameliorating a wound.

In some embodiments, the administering is to the wounded site.

Administering the composition to a specific site in the subject may be performed with any method known in the art. This may include with an applicator, in the form of a gel or cream, as well as on a scaffold, wrap or bandage.

As used herein, the terms “treatment” or “treating” of a disease, disorder or condition (e.g., a wound) encompasses alleviation of at least one symptom thereof, a reduction in the severity thereof, or inhibition of the progression thereof. Treatment need not mean that the disease, disorder or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, the treating or ameliorating comprises: increasing: the number of proliferating cells, including proliferation rate of epithelial cells and/or cells of the epidermis and/or dermis, such as hair follicle cells, the number of epidermal invaginations in the skin, or any combination thereof, reducing the surface area of the wound, or any combination thereof.

Methods for quantifying and/or determining epidermal invaginations, and de novo synthesis of hair follicles, would be apparent to one of ordinary skill in the art, and may include histology analysis, e.g., fluorescent (immunohistochemistry), such as exemplified herein.

Methods for quantifying and/or determining cell proliferation would be apparent to one of ordinary skill in the art, and may include gene expression analysis, PCR, western blot analysis, etc., using, for example, in the quantification of cell proliferation biomarkers, including, but not limited to Mcm2, Ki67, PCNA, and phosphorylated CREB, such as exemplified herein.

In some embodiments, hair follicle cells comprises hair follicle stem cells.

In some embodiments, the composition of the disclosure induces proliferation of an epithelial cell, a hair follicle stem cell, or both. In some embodiments, the composition of the disclosure increases the proliferation rate of an epithelial cell, a hair follicle stem cell, or both. In some embodiments, the composition of the disclosure increases the number of proliferating epithelial cells, hair follicle stem cells, or both.

In some embodiments, the increase is at least 5%, 10%, 35%, 50%, 80%, 100%, 150%, 270%, 400%, 650%, 800%, or 1,000% increase compared to a control cell, or any value and range therebetween. Each possibility represents a separate embodiment of the disclosure.

Models and methods for determining therapeutic relevancy in the context of wound healing and/or skin regeneration are common and would be apparent to one of ordinary skill in the art. Non-limiting example for such model includes, but is not limited to adult mouse skin during telogen (resting hair follicle stage; 8-weeks post-partum), and expression determination of biomarkers, such as, but not limited to, CB1, Keratin-15+, Ki67, pCREB^{S133}, CD104, as exemplified hereinbelow.

As used herein, the terms “skin regeneration” and “skin replenishment” are interchangeable.

Models and methods for determining therapeutic relevancy in the context of acne are common and would be apparent to one of ordinary skill in the art. A non-limiting example for such model includes but is not limited to the sebaceous gland-like organoid, as exemplified herein, and previously described in International Patent Application No. PCT/IL2019/051034, which is incorporated herein by reference in its entirety.

In another embodiment, the composition is administered by intravenous, intra-arterial, or intramuscular injection of a liquid preparation. In some embodiments, liquid formulations include solutions, suspensions, dispersions, emulsions, oils and the like. In one embodiment, the composition is administered intravenously, and is thus formulated in a form suitable for intravenous administration. In another embodiment, the composition is administered intra-arterially, and is thus formulated in a form suitable for intra-arterial administration. In another embodiment, the composition is administered intramuscularly, and is thus formulated in a form suitable for intramuscular administration.

Further, in another embodiment, the composition is administered topically to body surfaces, and is thus formulated in a form suitable for topical administration. Suitable topical formulations include gels, ointments, creams, lotions, drops and the like. For topical administration, the active ingredients disclosed herein, e.g., one or more cannabinoids, are combined with an additional appropriate therapeutic agent or agents, prepared, and applied as solutions, suspensions, or emulsions in a physiologically acceptable diluent with or without a pharmaceutical carrier.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, the composition is a suspension, a solution, or an emulsion in oily or aqueous vehicle, and contains a suspending, a stabilizing and/or a dispersing agent.

In some embodiments, a composition for parenteral administration includes aqueous solution of the active preparation in water-soluble form. Additionally, suspensions of the active ingredients, in some embodiments, are prepared as appropriate oily or water-based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contains suitable stabilizers or agents which increase the solubility of the active ingredients to allow for the preparation of highly concentrated solutions.

In another embodiment, the composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, further polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In some embodiments, the composition is formulated with a carrier. In some embodiments, the composition is encapsulated.

Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In one embodiment, the amount of a composition to be administered will be dependent on the subject being treated, the severity of the affliction, the manner of administration, the judgment of the prescribing physician, etc.

The dosage administered will be dependent upon the age, health, and weight of the recipient, kind of concurrent treatment, if any, frequency of treatment, and the nature of the effect desired.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if cach and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Cannabinoids and Uses Thereof in the Treatment of Inflammatory Skin Disease

TABLE 1
Extract compositions used
Extract composition (%) (w/w)

| | Infinity | White Widow |
|---|---|---|
| CA No. | (INF) | (WW) |
| CBDVA | n.a. | n.a. |
| CBDV | n.a. | 0.12 |
| CBDA | n.a. | n.a. |
| CBGA | n.a. | n.a. |
| CBG | 2.15 | 3.62 |
| CBD | 0.309 | 0.1836 |
| THCV | 0.51 | 0.4 |
| CBCV | n.a. | n.a. |
| CBN | 3.12 | 2.76 |
| THC | 74.02 | 74.16 |
| d8-THC | n.a. | n.a. |
| CBNA | n.a. | n.a. |
| CBL | n.a. | n.a. |
| CBC | 0.75 | 2.74 |
| THCA | n.a. | 0.11 |
| CBCA | n.a. | n.a. |
| Cannabicitran | 0.06 | 0.31 |

Not applicable, n.a.

TABLE 2
Cannabinoids (including their doses) use in in vivo and in vitro
assays in the context of psoriasis, as described herein.

| | In vivo | In vitro |
|---|---|---|
| INF | 1 mg/ml (0.1 mg | 1 mg/ml stock |
| (botanical) | per 20 g mouse) | (1 μg/ml treatment) |
| CBN | 0.031 mg/ml (0.0031 mg | 10 mM stock |
| (botanical) | per 20 g mouse) | (10 μM treatment) |
| CBC | 0.0075 mg/ml (0.00075 mg | 10 mM stock |
| (botanical) | per 20 g mouse) | (10 μM treatment) |
| THC | 0.74 mg/ml (0.074 mg | 10 mM stock |
| (botanical) | per 20 g mouse) | (10 μM treatment) |
| CBG | 0.0215 mg/ml (0.00215 mg | 10 mM stock |
| (botanical) | per 20 g mouse) | (10 μM treatment) |

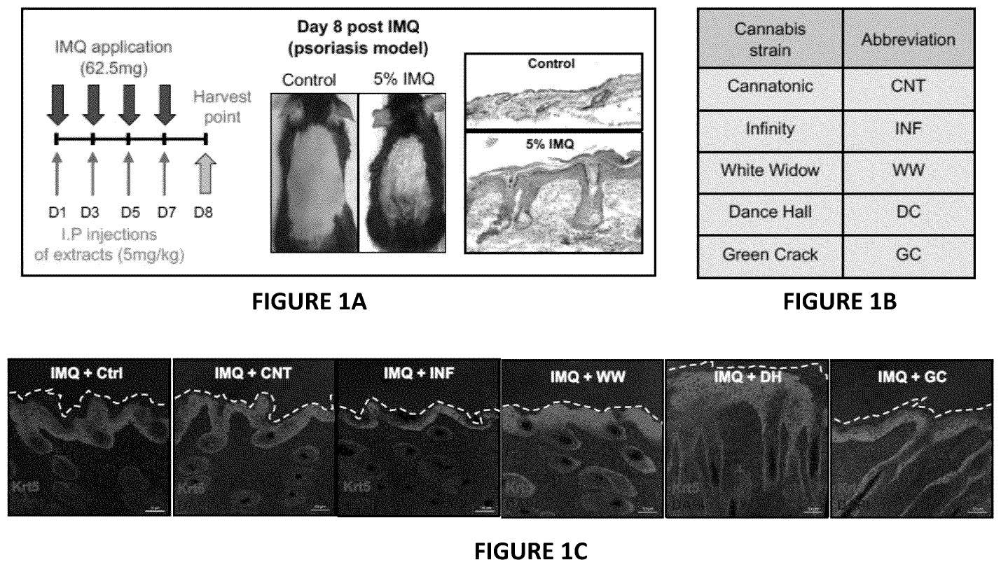

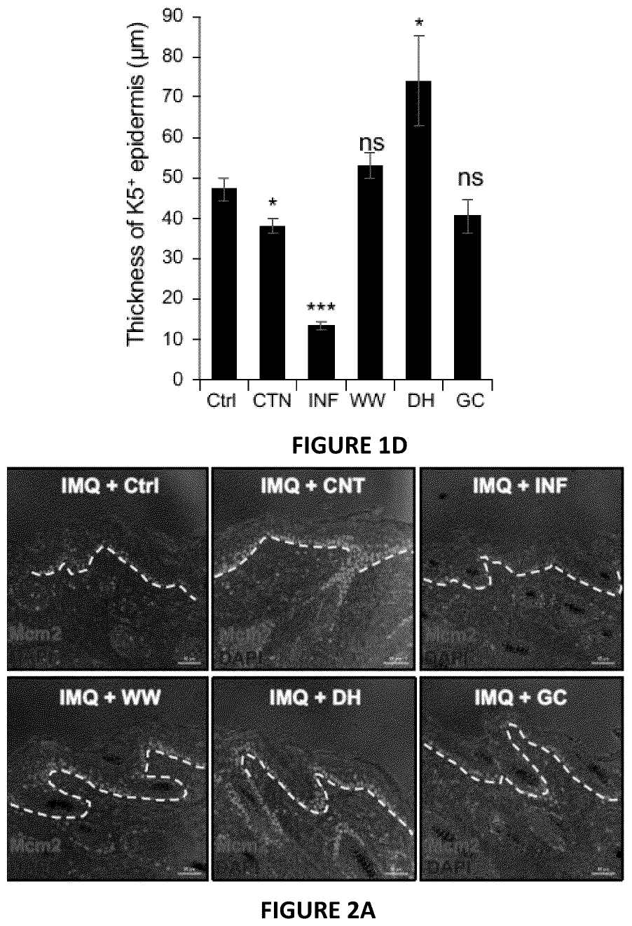

The therapeutic potentials of five different cannabis strains were determined (namely, Infinity (INF), White Widow (WW), Cannatonic (CNT), Dance Hall (DH), and Green Crack (GC)), utilizing an in vivo setting of skin pathology. For this aim, the topical 5%-Imiquimod (IMQ) model of psoriasis (FIGS. 1A-1B) was employed. In this model, 62.5 mg of IMQ cream is applied topically on depilated dorsal skins of mice (Fits et al., 2009) every second day concurrent with administration of extracts. Extracts were administered at 5 mg/kg dose via intraperitoneal (i.p.) injection.

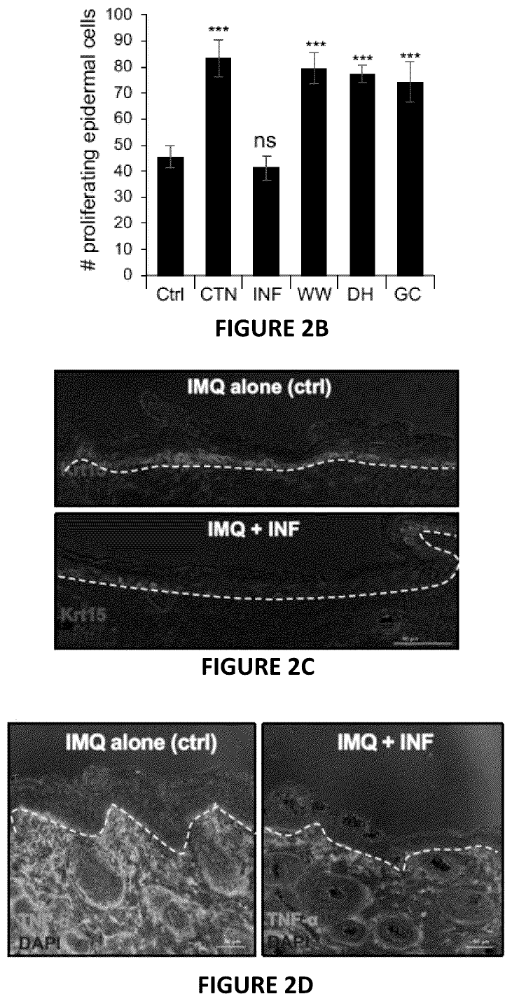

Strikingly, it was noted that mice treated with the extract Infinity (INF) displayed markedly less psoriatic disease symptoms by 8 days, including less visible skin lesions (scaling), very little thickening of the epidermis (FIGS. 1C-1D), as well as less proliferation, less inflammation, less TNF-alpha, and no defects in keratinocyte differentiation (FIG. 2). Mice treated with INF displayed no obvious clinical disease symptoms.

These findings suggest that INF, may be suitable for the treatment of a keratinocytes-related inflammatory skin disease or symptoms associated therewith, including psoriatic skin lesions.

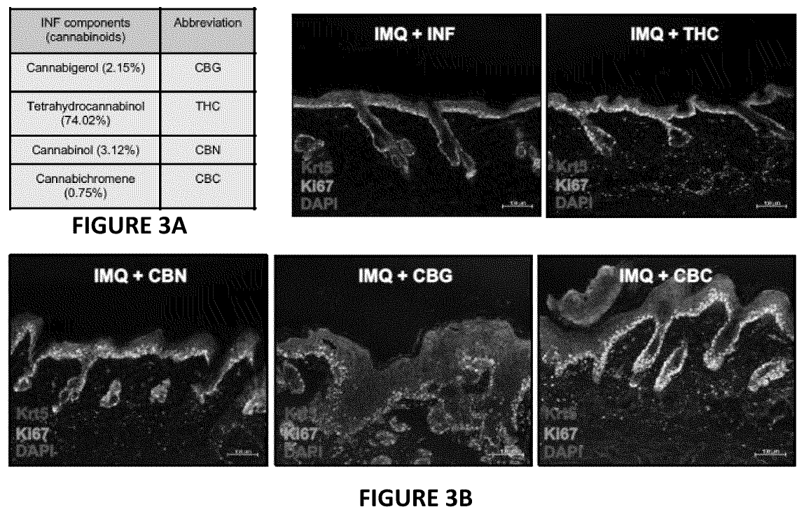

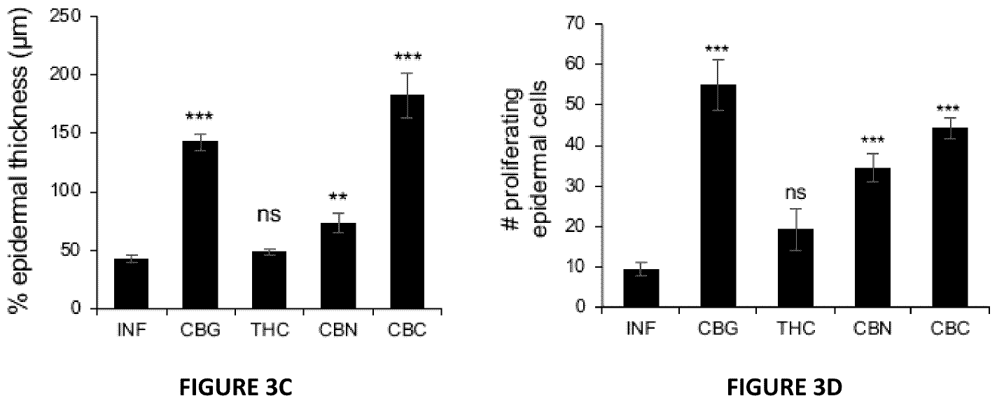

Examining the cannabinoid constituents of INF, the inventors next applied IMQ and treated mice with either control (vehicle only), the 5 mg/kg INF extract, 1.6 mg/kg CBN, 0.1 mg/kg CBG, 3.7 mg/kg THC, or 0.04 mg/kg CBC (FIG. 3A). In these analyses of the skin, it was shown that the effects of prevention of overgrowth of the epidermis and decreasing abnormal levels of proliferation were comparable for THC and the INF extract (FIGS. 3B-3D).

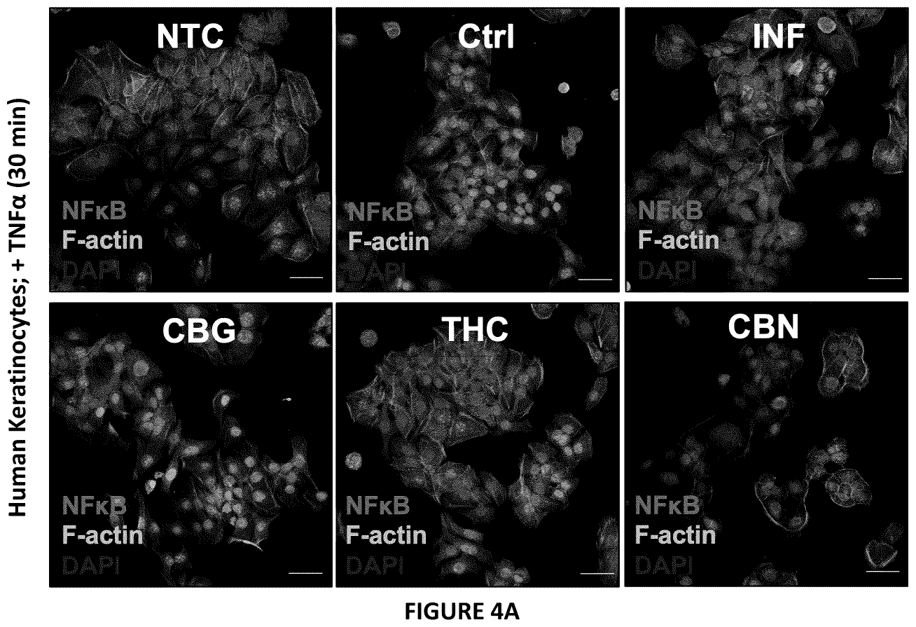

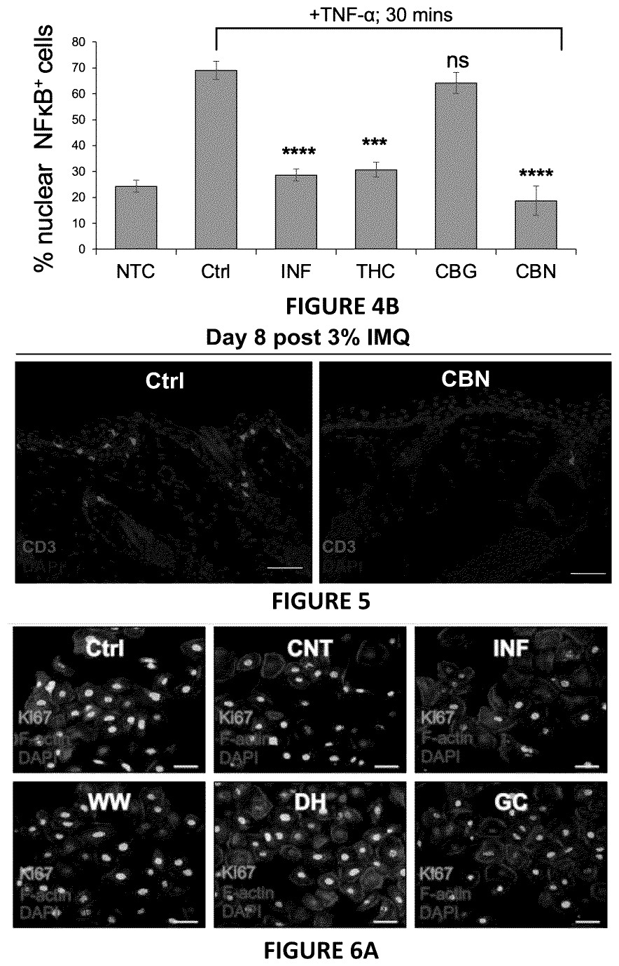

In order to examine the underlying mechanism, the inventors next examined whether improving health of the epidermis in psoriasis is dependent upon the NF-kappa-B (p65; NF-κB) transcription factor. NF-κB transcription factor is known to be active in psoriasis and high expression of its target genes leads to enhanced proliferation and inflammation (Goldminz et al., 2012). Human keratinocyte cells (HaCaT) were utilized. Cells were serum starved for 16 hours and then either non-treated (NTC) or administered with TNF-α along with either DMSO (Ctrl), Infinity (INF) extract, CBN, CBG, or THC at a concentration according to Table 2. Immunofluorescence was next performed for activated NF-κB using specific antibodies. The present data clearly indicate that, in response to TNF-α, the NF-κB (p65) transcription factor translocated to the nucleus. Notably, administration of CBG had no effect of NF-κB translocation, while the complete INF extract, THC, or CBN strongly inhibited the translocation of NF-kappa-B (p65) to the nucleus (FIG. 4A). CBN gave the best results of inhibition of NF-κB (p65) translocation (FIG. 4B).

The present results indicate that different cannabinoids affect NF-κB activation differently indicating that specific cannabinoid such as CBN can be utilized for treating a skin inflammatory disease, including a keratinocytes-related inflammatory disease, such as psoriasis. NF-κB is known to drive cytokine and elicit an immune response.

Further, the recruitment of CD3⁺ T cells to the epidermis was examined in 5% IMQ-treated skins. CD3⁺ T cells have been shown to infiltrate the epidermis in psoriatic skin lesions, which promotes inflammation (Brandt et al., 2007). The data show that CBN administration substantially reduced induced the recruitment of CD3⁺ T-cells, as was demonstrated by strong decrease in these cells' numbers (FIG. 5).

Therefore, it is concluded that while cannabinoids in general are highly similar and derived from a common precursor (e.g., are derivatives of one another), not all are effective or equally effective, as presented herein in the lack of activity of CBG, and the superior activity of CBN.

Taken together, these results indicated that INF extract in general, and particularly CBN, may serve as a powerful therapeutic tool for treating a keratinocyte-related inflammatory skin disease, inclusive of psoriasis.

### Example 2

### Cannabinoids in Treating Sebaceous Gland Proliferative Disease

Extract compositions included:

Extract composition (%) (w/w)

| CA No. | Infinity (INF) |
|---|---|
| CBDVA | n.a. |
| CBDV | n.a. |
| CBDA | n.a. |
| CBGA | n.a. |
| CBG | 2.15 |
| CBD | 0.309 |
| THCV | 0.51 |
| CBCV | n.a. |
| CBN | 3.12 |
| THC | 74.02 |
| d8-THC | n.a. |
| CBNA | n.a. |
| CBL | n.a. |
| CBC | 0.75 |
| THCA | n.a. |
| CBCA | n.a. |
| Cannabicitran | 0.06 |

For in vivo and in vitro assays in the context of sebaceous proliferative disease, as described herein, the inventors used the following cannabinoids (including their doses):

| | In vivo | In vitro |
|---|---|---|
| DH | — | 1 mg/ml stock |
| | | (1 μg/ml treatment) |
| GC | — | 1 mg/ml stock |
| | | (1 μg/ml treatment) |
| WW | — | 1 mg/ml stock |
| | | (1 μg/ml treatment) |
| CNT | — | 1 mg/ml stock |
| | | (1 μg/ml treatment) |
| INF | 1 mg/ml (0.1 mg | 1 mg/ml stock |
| | per 20 g mouse) | (1 μg/ml treatment) |
| CBN | 0.031 mg/ml (0.0031 mg | 10 mM stock |
| | per 20 g mouse) | (10 μM treatment) |
| CBC | 0.0075 mg/ml (0.00075 mg | 10 mM stock |
| | per 20 g mouse) | (10 μM treatment) |
| THC | 0.74 mg/ml (0.074 mg | 10 mM stock |
| | per 20 g mouse) | (10 μM treatment) |
| CBG | 0.0215 mg/ml (0.00215 mg | 10 mM stock |
| | per 20 g mouse) | (10 μM treatment) |

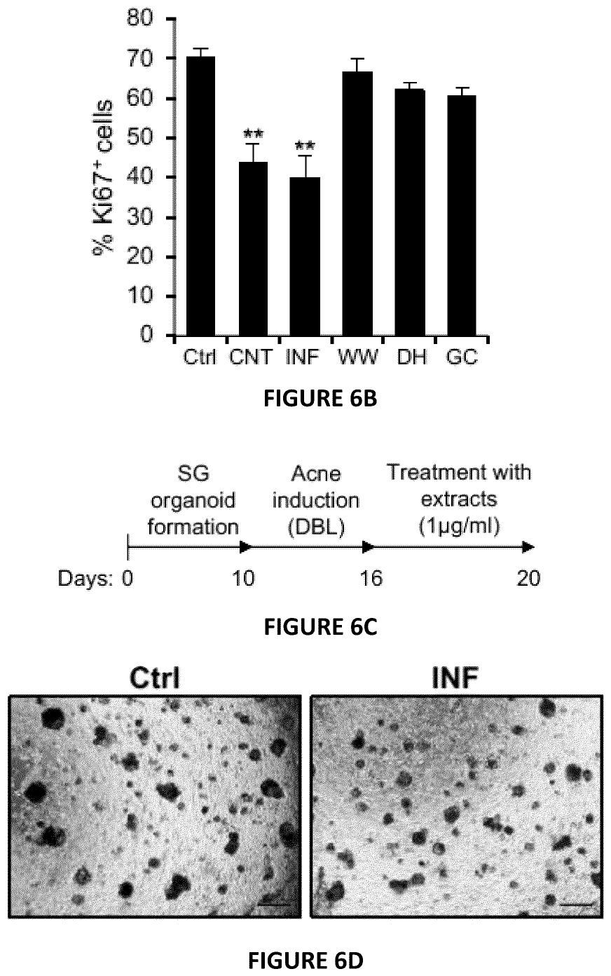

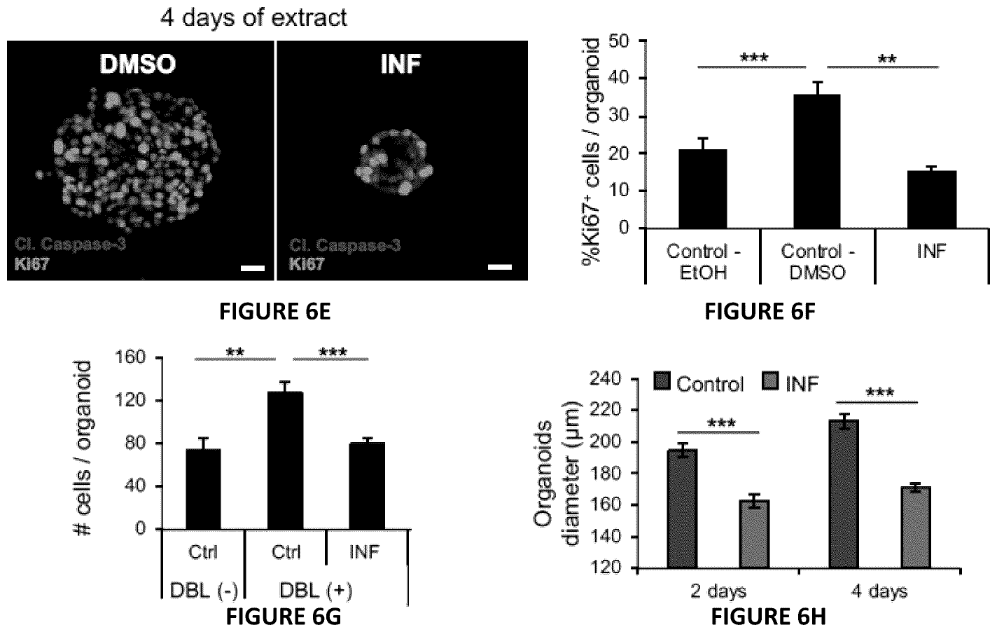

The therapeutic potentials of different cannabis strains were determined utilizing mouse primary sebaceous gland progenitors (Blimp1⁺) as well as an ex vivo sebaceous gland (SG) organoid platform for acne formation, which was previously established (see Feldman et al., 2019). As a first step, the inventors tested the effects of Green Crack (GC), Dance Hall (DH), Cannatonic (CNT), Infinity (INF) and White Widow (WW) in Blimp1⁺ sebaceous gland progenitors and determined the greatest decrease of proliferation in cells treated with INF (FIGS. 6A-6B). In the SG organoid model, basic symptoms of acne vulgaris (e.g., SG size, sebocyte hyperproliferation, increased sebum production) can be recapitulated through the administration of dihydrotestosterone (DHT) androgen, the PPAR-γ BRL-49653 (BRL) activator and linoleic acid (LIN; denoted DBL). (Feldman et al, 2019). In this model, culture medium of SG organoids contains 10⁻⁵ M of DHT, 10⁻⁶ M of BRL, 10⁻ M of LIN, or pure ethanol (solvent) as control for 6 days. After 6 days the medium is replaced with medium that contain extracts, which is replaced to normal medium after 4 days (FIG. 6C). In DBL-treated organoids treated with the extract Infinity (INF), organoids displayed decreased SG size, cell number per organoid and proliferation levels (% Ki67⁺ proliferating cells/organoid total number of cells) in contrast to the DBL-treated control (FIGS. 6D-6H). Of note, apoptotic cells (positive for cleaved Caspase-3) could still be detected after INF treatment (FIG. 6E).

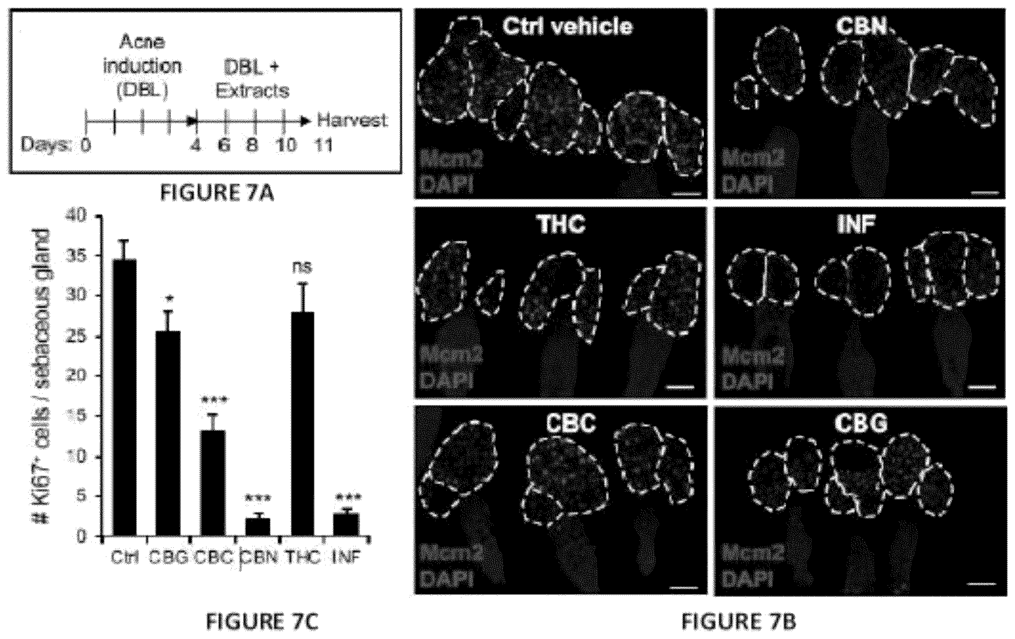

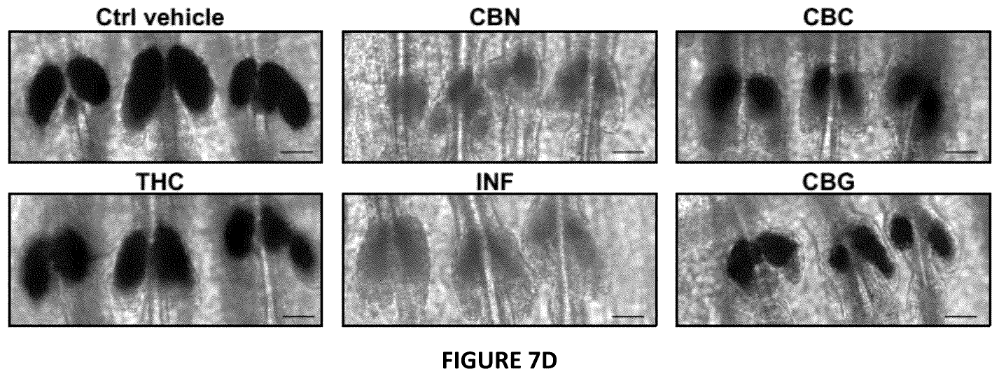

Next, in vivo studies was conducted to determine whether similar effects would be achieved. For this, DBL was applied in wild-type (WT; C57BL/6J) adult (8-week-old) male and female mice tail skins through intradermal injection at the tail base every second day for 10 days, which were treated from day 4 onwards with: 5 mg/kg INF extract, 1.6 mg/kg CBN, 0.1 mg/kg CBG, 3.7 mg/kg THC, or 0.04 mg/kg CBC (FIG. 7A). In the mouse tail, the SGs are highly proliferative and are easy to visualize making it an ideal system for studying SGs in their natural environment. Strikingly, CBN, alike INF, prevented hyperproliferation and sebum over-production (FIGS. 7B-7D).

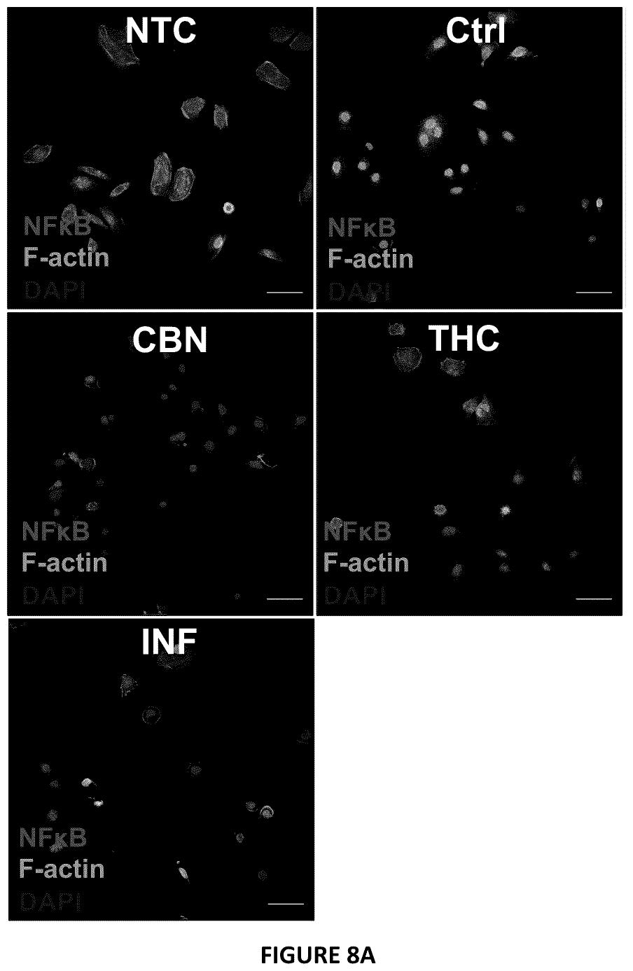

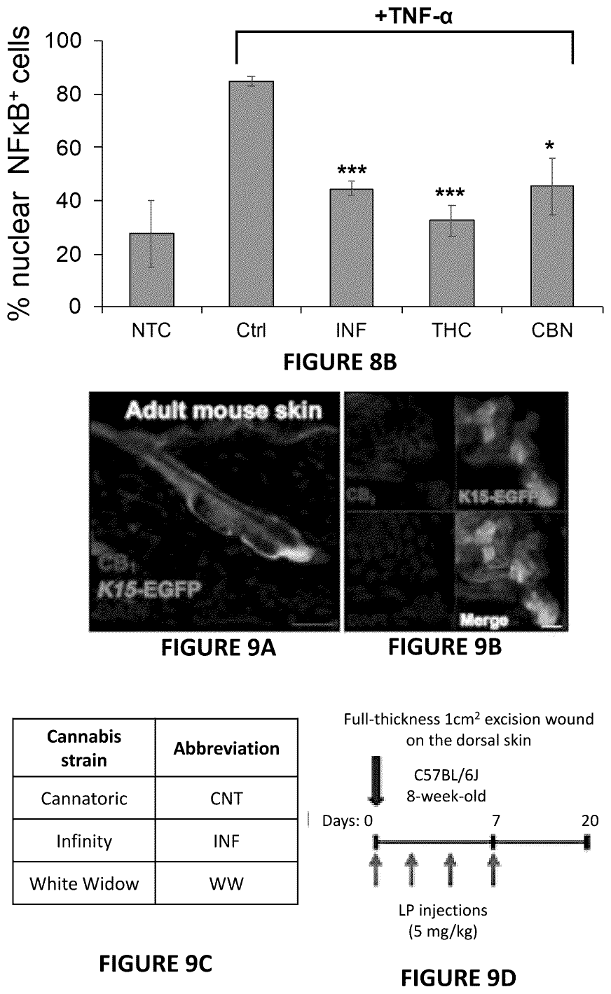

In order to examine the underlying mechanism, the inventors assessed the effects of INF, THC and CBN in acne and whether it is mediated through downregulation of NF-kappa-B (p65; NF-κB) signaling. NFκB transcription factor is known to be active in acne and high expression of its target genes leads to enhanced proliferation and inflammation (Kang et al., 2005). The inventors utilized mouse primary sebaceous gland progenitors (Blimp1⁺ sebocytes). Cells were serum starved for 16 hours and then either non-treated (NTC) or administered with TNF-α along with either DMSO (Ctrl), Infinity (INF) extract, CBN or THC (FIGS. 8A-8B). The inventors next performed immunofluorescence for activated NF-κB using specific antibodies. The present data clearly indicate that in response to TNF-α the NF-κB (p65) transcription factor translocates to the nucleus in Blimp1⁺ sebocytes. Notably the INF extract, THC and CBN strongly inhibited the translocation of NFκB (p65) to the nucleus (FIGS. 8A-8B). These findings suggest the potential use of CBN in treating acne by blocking NFκB activation, including extracts of WW, and INF, comprising same.

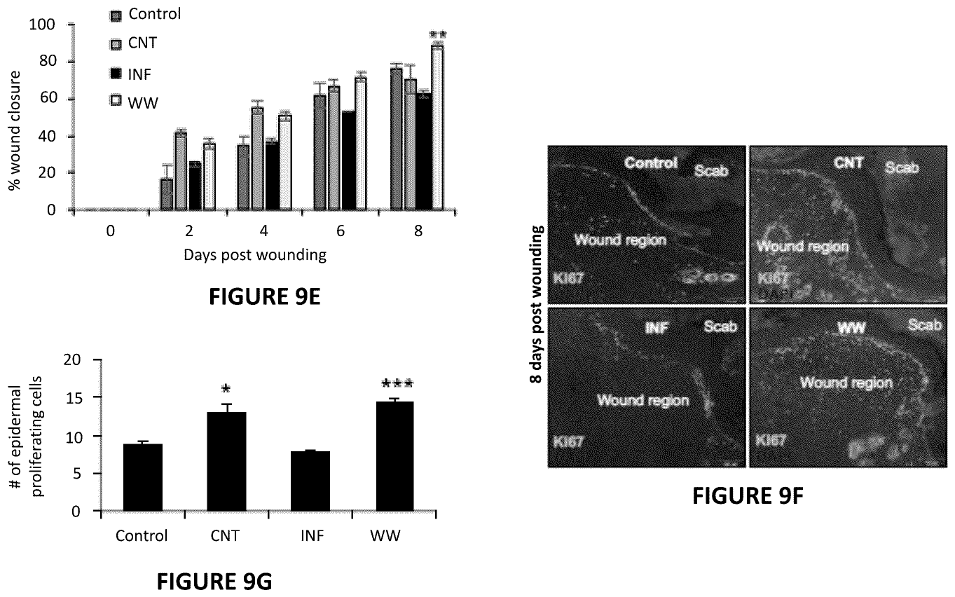

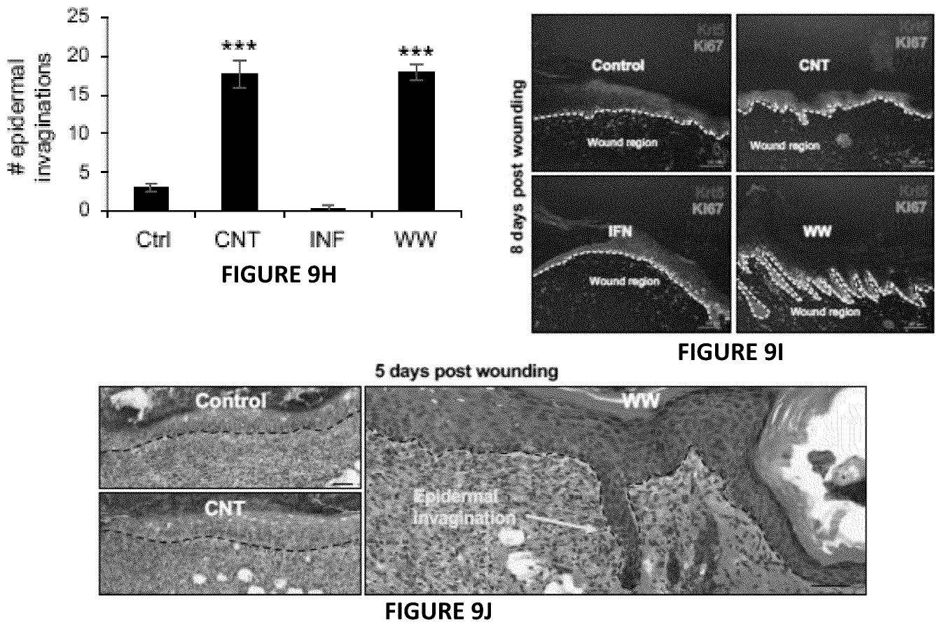

FIGS. 9A-9J include fluorescence micrographs, a table, a scheme of a non-limiting study design, vertical bar graphs, and histological micrographs showing that cannabis strain ‘White widow’ (WW) enhances wound healing and regeneration. (9A) Immunostaining of adult Krt15-EGFP telogenic (8-week-old) dorsal skin with CB1 receptor. EGFP marks Krt15⁺ hair follicle stem cells (HFSCs). (9B) Zoom-in of HFSCs showing single channels. (9C) Table outlining cannabis strains. (9D) A non-limiting schematic outline of wound infliction and treatment regimen. (9E) Percentage of wound coverage (vs. initial wound size) of control, CNT, INF, and WW-treated wounds. (9F) Immunostaining of wound sections after 8 days post wounding with proliferation marker Ki67. (9G) Quantification of Ki67⁺ cells in the wound site at 8 days. Hematoxylin and Eosin stain of treated wound sections at 5 days PWI. (9H) Wound sections at 8 days immunostained with Krt5 and Ki67. (9I) Quantification of number of epidermal invaginations within the wound site. (9J) H&E of control, CNT, and WW-treated wound sections at 5 days PWI. *p<0.05, **p<0.01, ***p<0.001 as determined by two-tailed Student's T test. White dotted lines demarcate epidermis/dermis border. n<3 in all experiments.

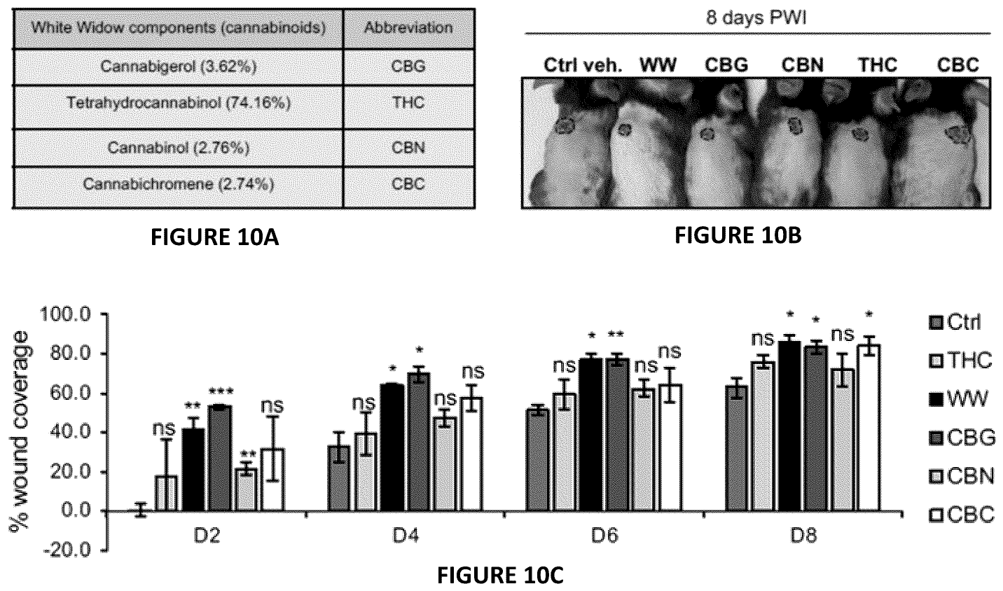

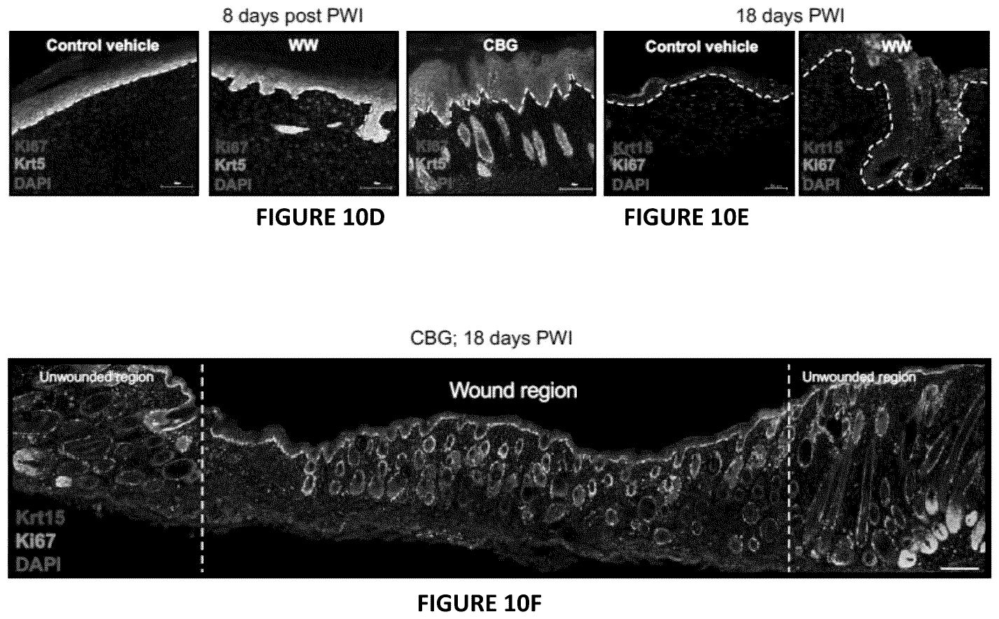

FIGS. 10A-10F include a table, an image, a vertical bar graph, and fluorescence micrographs showing that the WW constituent cannabigerol (CBG) improves healing and regeneration over other cannabinoids. (10A) Table outlining cannabinoid composition of WW. (10B) Representative photograph of wounded treated mice after 8 days post wounding. (10C) Quantification of dorsal wound closure. (10D) Immunostaining of Ki67 and Krt5 in wound sections after 8 days. (10E-10F) Immunostaining of wound sections after 18 days post wound infliction with Krt15 and Ki67. ns=no significance, *p<0.05, **p<0.01, and ***p<0.001 compared to the control as determined by two-tailed Student's t test. Error bars show S.E.M. White dotted lines demarcate epidermis/dermis border. n<3 in all experiments.

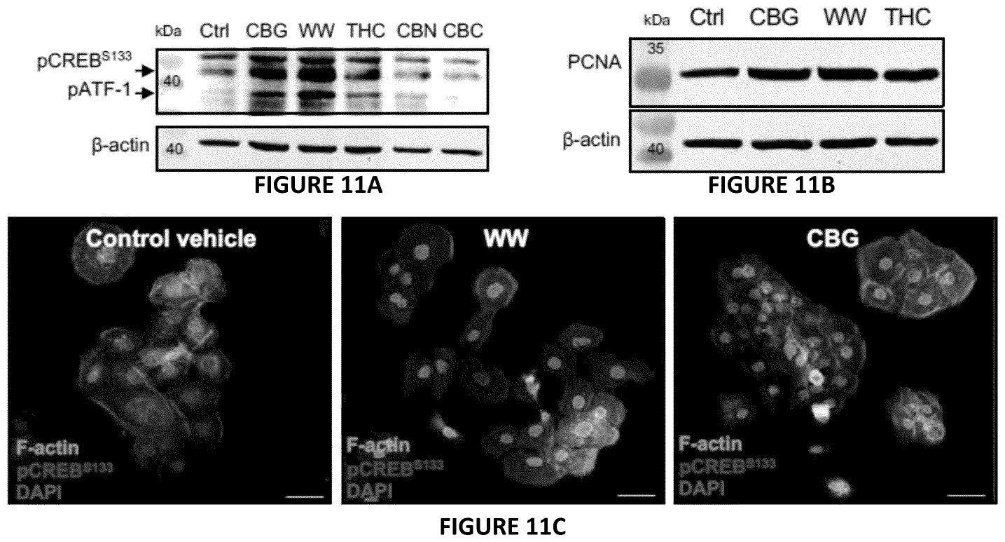

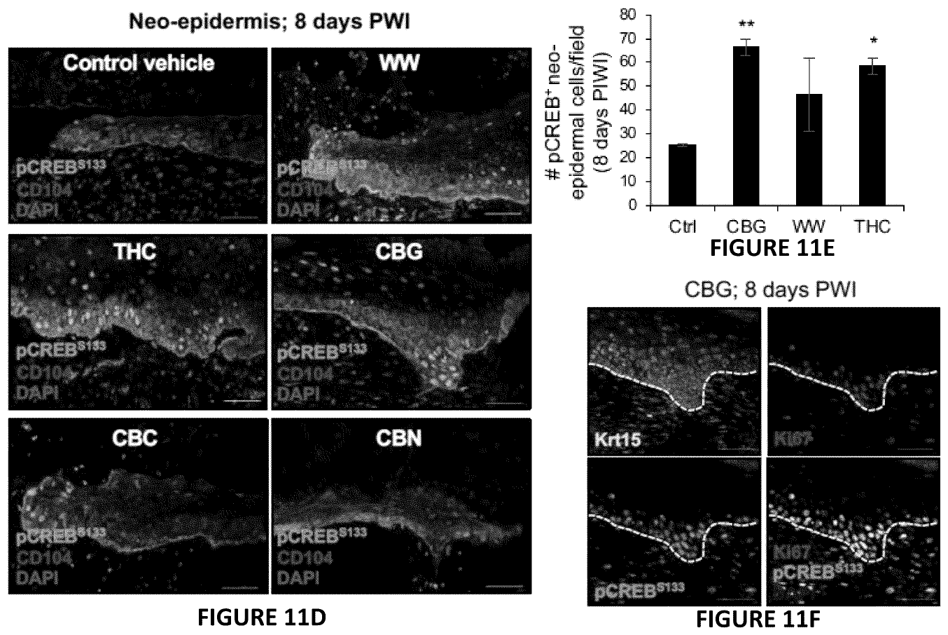

FIGS. 11A-11F include micrographs, fluorescence micrographs and a vertical bar graph, showing that white widow (WW) extract and CBG drive CREB activation in vitro. and proliferation in vitro and in vivo. (11A) HaCaT keratinocytes were serum starved for 16 hours and induced with WW extract or CBG. At 30 minutes post-stimulation cells were harvested and subjected to immunofluorescence showing pCREBS133 and F-actin expression. Scale bars: 50 μm. (11B) WW extract and CBG drive CREB activation in vitro. (11C) WW extract and CBG drive keratinocyte proliferation in vitro. (11D) WW extract and CBG drive CREB activation in wounds in vivo. Neo-epidermis of wounded and treated mice after 8 days post wound infliction (PWI) showing phosphorylated CREB. CD104 marks neo-epidermal cells (e.g., basement membrane marker). (11E) quantification of pCREB+ neo-epidermal cells. Scale bars: 50 μm. ***p<0.001, *p<0.05, ns=no significance. (11F) CBG drives CREB activation in proliferating cells. Wounds were harvested and immunostained for pCREB^{s133} and Ki67 expression. Scale bars: 50 μm.

### Example 3

### Cannabinoids Use in Skin Regeneration

Extract compositions included:

Extract composition (%) (w/w)

| | Infinity | White Widow |
|---|---|---|
| CA No. | (INF) | (WW) |
| CBDVA | n.a. | n.a. |
| CBDV | n.a. | 0.12 |
| CBDA | n.a. | n.a. |
| CBGA | n.a. | n.a. |
| CBG | 2.15 | 3.62 |
| CBD | 0.309 | 0.1836 |
| THCV | 0.51 | 0.4 |
| CBCV | n.a. | n.a. |
| CBN | 3.12 | 2.76 |
| THC | 74.02 | 74.16 |
| d8-THC | n.a. | n.a. |
| CBNA | n.a. | n.a. |
| CBL | n.a. | n.a. |
| CBC | 0.75 | 2.74 |
| THCA | n.a. | 0.11 |
| CBCA | n.a. | n.a. |
| Cannabicitran | 0.06 | 0.31 |

For in vivo and in vitro assays in the context of wound, regenerating skin, alopecia, and any disorder, condition, or symptom associated therewith, as described herein, the inventors used the following cannabinoids (including their doses):

| | In vivo | In vitro |
|---|---|---|
| WW | 1 mg/ml (0.1 mg | 1 mg/ml stock |
| | per 20 g mouse) | (1 μg/ml treatment) |
| CBN | 0.031 mg/ml (0.0031 mg | 10 mM stock |
| | per 20 g mouse) | (10 μM treatment) |
| CBC | 0.0075 mg/ml (0.00075 mg | 10 mM stock |
| | per 20 g mouse) | (10 μM treatment) |
| THC | 0.74 mg/ml (0.074 mg | 10 mM stock |
| | per 20 g mouse) | (10 μM treatment) |
| CBG | 0.0215 mg/ml (0.00215 mg | 10 mM stock |
| | per 20 g mouse) | (10 μM treatment) |

Initially, adult mouse skin was utilized during telogen (resting hair follicle stage; 8-weeks post-partum) to examine the expression of CB1. The expression of Cannabinoid receptor type 1 (CB1) was determined throughout the epidermis as well as the hair follicle (FIGS. 9A-9B). The expression of CB1 was detected in constitutively EGFP (enhanced green fluorescence protein)-expressing Keratin-15⁺ (K15-EGFP⁺) hair follicle stem cells, which play key roles in epidermal wound healing as well as adult hair follicle regeneration.

Three cannabis strains (FIG. 9C), Cannatonic (CNT), Infinity (INF) and White Widow (WW) were examined for their regenerative and wound healing potentials in vivo.

To this end, 1 cm² whole-thickness dorsal wounds were inflicted on male or female 8-week-old wildtype (WT; C57BL/6J background) mice (n=at least 3 mice per group) and the wounded mice were treated with 5 mg/kg of each strain via intraperitoneal (IP) injection every two days (FIG. 9D). Dorsal wound coverage was measured by determining wound size (%) relative to the initial wound size. Notably, mice treated with the WW strain exhibited faster wound closure (FIG. 9E). Examining the wound sections, the inventors found that WW treatment led to enhanced levels of proliferation within the wound site (FIGS. 9F-9H). Strikingly, at 8 days post wounding the inventors detected large epidermal invaginations within the WW-treated wound site (FIG. 9H), which were very pronounced by 8 days and were largely absent in the control (FIGS. 9H-9I). Of note, the inventors could detect epidermal invaginations in the WW-treated wound in as little as 5 days post wounding, which was absent in the control and CNT-treated wounds (FIG. 9J).

Next, the inventors sought to understand whether any single cannabinoid component of the WW strain could potentiate the WW-induced phenotypes (FIG. 10A). For this, male and female mice (8-weeks-old) on WT (wild type; C57BL/6J) background were administered IP injections of cannabigerol (CBG; 0.11 mg/kg), tetrahydrocannabinol (THC; 3.7 mg/kg), cannabinol (CBN; 0.155 mg/kg), or cannabichromene (CBC; 0.0375 mg/kg), and dorsal wound was inflicted (n=at least 3 mice per group). Cannabinoid solutions are prepared by dissolving pure cannabinoids in 100% ethanol (EtOH) to a stock concentration of 100 mg/ml. Further dilution was achieved by preparing a 1:1:18 working solution of 100% EtOH:Cremophor:1×PBS. 100 μl was administered via IP injection to each mouse every second day starting from the day of wound infliction.

Strikingly, mice treated with CBG and, to some extent, with THC, healed comparably to the WW strain (FIGS. 10B-10C). When examining the tissue at 8 days post wounding, it was noted that considerably more epidermal invaginations in skins treated with CBG (FIG. 10D) and by 18 days a striking number of regenerated hair follicles in the CBG-treated wounds (FIGS. 10E-10F). These results suggested that CBG alone may serve as a useful therapeutic tool in promoting wound healing and improving regeneration over some cannabinoids. In some incidences, it might be beneficial to combine CBG with other cannabinoids to further the wound healing property and other skin regeneration benefits of CBG.

In order to examine the underlying mechanism, the inventors next examined whether increased repair and proliferation is facilitated via the CAMP response element binding protein (CREB). First, the inventors utilized human keratinocyte cells (HaCaT). Cells were starved (serum-free) for 16 hours and then administered with Dimethyl sulfoxide (DMSO; carrier/solvent control), White Widow (WW) extract or cannabigerol (CBG). Next, the inventors performed immunofluorescence for activated phosphorylated CREB using specific antibodies. The data clearly indicates that in response to administration of either the WW extract or CBG, CREB becomes highly phosphorylated (denoted pCREB^{S133}) and translocates to the nucleus (FIG. 11A).

In order to verify these findings, the inventors next performed western blot analysis of treated cells (as outlined above). The inventors found that administration of DMSO or other cannabinoids including THC, CBN and CBC had only minimal (if any) effect on the levels of active pCREB^{S133} (known to recognize the cAMP Response Element (CRE) and serve as a transcription factor) or ATF1 (activated transcription factor 1 of the CREB family; FIG. 11B). In contrast, both the WW extract and CBG resulted in high phosphorylation of CREB and ATF (FIG. 11B) corroborating the herein disclosed immunofluorescence results (FIG. 11A). Furthermore, the WW extract and CBG resulted in increased proliferation as indicated by the proliferation marker PCNA (FIG. 11C).

Thereafter, the inventors examined whether CREB is phosphorylated in healing wounds. The inventors performed full-thickness excisional wounds (as described above) and harvested wounds at 8 days post wound infliction. The present immunofluorescence results clearly indicate that administration of either the WW extract or CBG leads to a dramatic increase in activated pCREB^{S133} in the epidermis adjacent to the wound (migrating epithelial edge; FIGS. 11D-11E).

Furthermore, the inventors show that in CBG-treated wounds, activated p-CREB was co-expressed with proliferative markers and localized at epidermal invasions (placodes) which gave rise to regenerated hair follicles (FIG. 11F).

Taken together, these results indicate that CBG may serve as a powerful therapeutic tool for increasing wound healing and improving regeneration.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method for treating any one of: a wound, regenerating skin, alopecia, and any disorder, condition, or symptom associated therewith, in a subject in need thereof, the method comprising administering to said subject a therapeutically effective amount of a pharmaceutical composition comprising one to five cannabinoids, wherein said one to five cannabinoids comprises CBG, thereby treating a wound, regenerating skin, or both, in the subject.

2. The method of claim 1, wherein said one to five cannabinoids is CBG; (ii) said pharmaceutical composition further comprises tetrahydrocannabinol (THC), and optionally wherein said THC is (−)-Δ⁹-trans-tetrahydrocannabinol (Δ⁹-THC), said CBG and THC are present in said composition in a weight per weight ratio of about 1:10 (w/w) to 1:35 (w/w), or both; and (iii) both (i) and (ii).

3.-5. (canceled)

6. The method of claim 1, wherein said pharmaceutical composition is devoid of CBN and CBC.

7. The method of claim 1, wherein one or more of said cannabinoids is present as a highly purified extract of Cannabis.

8. The method of claim 1, wherein one or more of said cannabinoids is a synthetically produced cannabinoid.

9. The method of claim 1, wherein said treating comprises reducing: the area size of said wound, increasing the number of skin invaginations, increasing the number of skin proliferating cells, increasing the proliferation rate of skin cells, inducing hair follicle growth or regeneration, inhibiting, or reducing scarring, or any combination thereof, in said subject.

10. A method for treating a subject afflicted with an inflammatory skin disease, the method comprising administering to said subject a therapeutically effective amount of a pharmaceutical composition comprising one to five cannabinoids, wherein said one to five cannabinoids comprises: CBN, thereby treating the subject afflicted with an inflammatory skin disease.

11. The method of claim 10, wherein any one of: (i) said one to five cannabinoids is CBN; (ii) said one to five cannabinoids comprises THC, and optionally wherein said THC is (−)-Δ⁹-trans-tetrahydrocannabinol (Δ⁹-THC), said pharmaceutical composition comprises CBN and THC in a weight per weight ratio (w/w) ranging from 1:10 to 1:40, or both; and (iii) both (i) and (ii).

12.-14. (canceled)

15. The method of claim 10, wherein said pharmaceutical composition is devoid of any one of: CBG, CBC, and both.

16. The method of claim 10, wherein one or more of said cannabinoids is present as a highly purified extract of Cannabis.

17. The method of claim 10, wherein one or more of said cannabinoids is a synthetically produced cannabinoid.

18. The method of claim 10, wherein said treating comprises inhibiting or reducing: NF-κB migration from the cytosol to the nucleus, NF-κB signaling, % of nuclear NF-κB, or any combination thereof, in keratinocytes of said subject.

19. The method of claim 10, wherein said inflammatory skin disease comprises a keratinocytes-related inflammatory disease.

20. The method of claim 10, wherein said inflammatory skin disease is selected from the group consisting of: psoriasis, allergic contact dermatitis, and atopic dermatitis.

21. A method for treating a subject afflicted with a sebaceous gland proliferative disease, comprising administering to said subject a therapeutically effective amount of a pharmaceutical composition comprising one to five cannabinoids, wherein said one to five cannabinoids comprises: CBN, CBG, CBC, THC, or any combination thereof, thereby treating the subject afflicted with a sebaceous gland proliferative disease.

22. The method of claim 21, wherein any one of: (i) said one to five cannabinoids is CBN; (ii) said pharmaceutical composition comprises CBN, CBG, CBC, and THC; (ii) said pharmaceutical composition comprises any one of: (a) CBN and CBG in a weight per weight ratio (w/w) ranging from 2:1 to 1:2; (b) CBN and CBC in a w/w ranging from 5:1 to 1:1; (c) CBG and CBC in a w/w ranging from 4:1 to 2:1; (d) CBN and THC in a w/w ranging from 1:5 to 1:35; or (e) any combination of (a) to (d); (iii).

23.-24. (canceled)

25. The method of claim 21, wherein one or more of said cannabinoids is present as a highly purified extract of Cannabis or is a synthetically produced cannabinoid.

26. (canceled)

27. The method of claim 21, wherein said treating comprises reducing: the number, rate, or both, of proliferating sebaceous cells, the size, growth rate, or both of sebaceous glands, sebum amount, production rate, or both, or any combination thereof, in said subject.

28. The method of claim 21, wherein said sebaceous gland proliferative disease is a hyperproliferative disease.

29. The method of claim 21, wherein said sebaceous gland proliferative disease is selected from the group consisting of: acne, sebaceous carcinoma, sebaceous hyperplasia, seborrhoea, seborrhoeic dermatitis, sebopsoriasis, sebaceous adenoma, nevus sebaceous, or phymatous rosacea.

30.-38. (canceled)
